(19) United States
(12) Patent Application Publication
(10) Pub. No.: US 2011/0300222 A1
(43) Pub. Date: Dec. 8, 2011

(54) LUMINESCENT POROUS SILICON NANOPARTICLES, METHODS OF MAKING AND USING SAME

(75) Inventors: Michael J. Sailor, La Jolla, CA (US); Luo Gu, La Jolla, CA (US); Ji-Ho Park, Daejeon (KR)

(73) Assignee: THE REGENTS OF THE UNIVERSITY OF CALIFORNIA, Oakland, CA (US)

(21) Appl. No.: 13/202,271

(22) PCT Filed: Feb. 20, 2010
(86) PCT No.: PCT/US2010/024848

§ 371 Date: Aug. 18, 2011

Related U.S. Application Data
(60) Provisional application No. 61/154,333, filed on Feb. 20, 2009.

(51) Int. Cl. A61K 9/16; C12Q 1/02; C01B 33/12; B32B 5/16; A61K 9/00; A61K 47/00; B82Y 40/00; B82Y 5/00

(52) U.S. Cl. 424/493; 424/400; 424/490; 435/29; 514/770; 423/335; 428/402; 977/700; 977/895; 977/927

(57) ABSTRACT

The disclosure relates to imaging agents and drug delivery systems.

### CROSS REFERENCE TO RELATED APPLICATIONS

This application claims priority under 35 U.S.C. §119 from Provisional Application Ser. No. 61/154,333, filed Feb. 20, 2009, the disclosure of which is incorporated herein by reference.

### STATEMENT AS TO FEDERALLY SPONSORED RESEARCH

The U.S. Government has certain rights in this disclosure pursuant to Grant Nos. 5-R01-CA124427, U54 CA 119335 and SUB 5710002094 awarded by the National Institutes of Health.

### TECHNICAL FIELD

The invention relates to delivery systems and, more particularly, to a device that can deliver preprogrammed quantities of a composition over time without the need for external power or electronics.

### BACKGROUND

Organic molecule-based nanoparticles generally require the addition of a molecular tag in order to allow in vivo monitoring by fluorescence. Although CNT, GN, and QD have demonstrated potential for in vivo imaging due to their unique optical properties, clinical translation has been impeded due to concerns regarding the biodegradability of such materials, the toxicity of degradation by-products, or the toxic structural characteristics of the nanomaterials themselves. Although efficient renal clearance can mitigate toxic effects, optimized formulations can leave significant residual heavy metals or other toxic constituents in MPS organs. Furthermore, the hydrodynamic size required for renal clearance (<5.5 nm) may be too small to allow the incorporation of functional components such as multivalent targeting ligands, and rapid renal excretion reduces the time available to the nanomaterial to perform its function.

### SUMMARY

The disclosure provides luminescent porous silicon nanoparticles (LPSiNP) that can carry a drug payload and whose intrinsic near-infrared (NIR) photoluminescence allows monitoring of both accumulation and degradation in vivo. Furthermore, in contrast to most optically active nanomaterials (e.g., carbon nanotubes (CNT), gold nanoparticles (GN), and quantum dots (QD)), LPSiNP self-destruct into renally cleared components in a relatively short period of time with no or little evidence of toxicity. For example, the LPSiNPs can be used for tumor imaging using dextran-coated LPSiNP (D-LPSiNP).

The disclosure provides porous silicon nanostructures with intrinsic NIR luminescence can be used for in vivo monitoring, they can be loaded with therapeutics, and they can be engineered to resorb in vivo into benign components that clear renally within specific timescales.

The disclosure provides a biodegradable porous nanostructure comprising silicon material, an emission spectra of about 500 to about 1000 nm and an excitation spectra between about 290-700 nm by single photon excitation or about 600-1200 nm by two photon excitation. In one embodiment, the silicon material comprises a silicon dioxide material. In another embodiment, the biodegradable porous nanostructure comprises a particulate size of between about 0.05 μm and 100 μm. In yet another embodiment, the biodegradable porous nanostructure can be characterized as non-toxic. In one embodiment, the biodegradable porous nanostructure is coated or encapsulated within a polymeric material. In a further embodiment, the polymeric material is selected from the group, but is not limited to, dextran, polyethylene glycol, a lipid, chitosan, zein, polylactic acid, polyglycolic acid, collagen, fibrin, co-polymers of polylactic acid and polyglycolic acid, and co-polymers of dextran and polylactic acid. In a specific embodiment, the polymeric material is dextran. In yet another embodiment, the biodegradable porous nanostructure further comprises a therapeutic drug.

The disclosure also provides a method of preparing a biodegradable imaging agent comprising electrochemically etching a p-type silicon wafer; lifting off a porous film from the silicon wafer substrate; fractionating the porous film to generate nanostructures; activating the nanostructure in an aqueous buffer. Such an aqueous buffer that may optionally contain chemical oxidizing agents of oxidizing power sufficient to convert Si to SiO₂. In one embodiment, the aqueous buffer comprises a borate solution. In yet another embodiment, the imaging agent comprises an emission spectra of about 500 to about 1000 nm and an excitation spectra between about 290-700 nm by single photon excitation or about 600-1200 nm by two photon excitation. In a further embodiment, the imaging agent further comprises loading a drug or agent into the pores of the nanostructure. In yet another embodiment, the method further comprising adsorbing a biocompatible agent to the nanostructure to increase the half-life or circulatory time in vivo.

The disclosure also provides a method of making a biodegradable porous nanostructure comprising: electrochemically etching a p-type silicon wafer; obtaining a free-standing hydrogen-terminated porous silicon film by removing the porous silicon nanostructure from the crystalline silicon substrates; fracturing the free-standing hydrogen-terminated porous silicon film to obtain a mixture of nanoporous materials of differing sizes; filtering or size selecting the fractured porous material to obtain a desired size fractionated nanoporous material; and activating the size fractionated nanoporous material by incubating the material in an aqueous buffer that is oxidizing or neutral to basic to obtain a luminescent porous silicon nanoparticle (LPSiNP). In one embodiment, the electrochemical etching is by application of a constant current density of about 200 mA/cm² for about 150 s in an aqueous HF/ethanol electrolyte. In another embodiment, the freestanding film is obtained by application of a current pulse of about 4 mA/cm² for 250 s in an aqueous HF/ethanol electrolyte. In yet another embodiment, the freestanding hydrogen-terminated porous silicon film is fractured by sonication. In one embodiment, the filtering comprising passing the nanoporous material through a 0.22 to 0.45 μm filtration membrane. In yet another embodiment, the activating comprises incubating the size fractionated nanoporous material in an aqueous buffer selected from the group consisting of an aqueous borate buffer, a phosphate buffered saline, and sodium hydroxide. In yet another embodiment, the activating comprises incubating the size fractionated nanoporous material in deionized water for approximately 2 weeks. In one embodiment, the method further comprises physically absorbing dextran to the LPSiNP or covalently attaching a polymeric material. In yet another embodiment, the method further comprises loading a therapeutic drug into the pores of the LPSiNP.

The disclosure also provides a pharmaceutical composition comprising a pharmaceutically acceptable carrier and a biodegradable porous nanostructure of the disclosure.

The disclosure provides a composition comprising: a biodegradable porous nanostructure comprising silicon, a plurality of pores and comprising an emission spectra of about 500 to about 1000 nm and an excitation spectra between about 290-700 nm by single photon excitation or about 600-1200 nm by two photon excitation; and a drug or biologically active material within the pores. In one embodiment, the biodegradable porous nanostructure further comprises a polymeric coating the increases the half-life or circulatory time of the biodegradable porous nanostructure in vivo.

The disclosure also provides a method of imaging a tissue, cell, or tumor comprising administering to a tissue, cell, or subject a LPSiNP of the disclosure and contacting the tissue, cell or subject with an excitation energy and measuring an emission spectra.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-F shows characterization of LPSiNPs. a, Schematic diagram depicting the structure and in vivo degradation process for the (biopolymer-coated) nanoparticles used in this study. b, SEM image of LPSiNPs (the inset shows the porous nanostructure of one of the nanoparticles). The scale bar is 500 nm (50 nm for the inset). c, Photoluminescence emission and absorbance spectra of LPSiNPs. Photoluminescence is measured using ultraviolet excitation (λ=370 nm). d, Appearance of silicon in solution (by ICP-CES) and photoluminescence intensity (λₑₓ=370 nm and λₑₙ=maximum peak intensity at each time point) from a sample of LPSiNPs (50 μg/ml) incubated in PBS solution at 37 C as a function of time. e, Release profile depicting percent of DOX from DOX-LPSiNPs released into a PBS solution as a function of time at 37° C. Data were obtained by filtering out DOX-LPSiNPs from the solution at each time point using a centrifugal filter and measuring the fluorescence intensity of free DOX left in solution (λₑₙ=590 nm; λₑₓ=480 nm). Error bars indicate s.d. f, Cytotoxicity of DOX-LPSiNPs, bare LPSiNPs and free DOX towards MDA-MB-435 human carcinoma cells, quantified by the MTT assay. The cells were incubated with the samples for 48 h.

FIGS. 2A-D shows biocompatibility and biodegradability of luminescent porous Si nanoparticles (LPSiNP). (a) In vitro cytotoxicity of LPSiNP towards HeLa cells, determined by Calcein assay. LPSiNP at the indicated concentrations were incubated with cells for 48 h. (b) In vivo biodistribution and biodegradation of LPSiNP over a period of 4 weeks in mouse. Aliquots of LPSiNP were intravenously injected into the mouse (n=3 or 4, dose=20 mg/kg). The silicon concentration in the organs was determined at different time points after injection using ICP-OES. Error bars indicate s.d. (c) Change in body mass of mice injected with LPSiNP (n=3, dose=20 mg/kg) compared with PBS control (n=4). There is no statistically significant difference in the mass change between control (PBS) and LPSiNP over a period of 4 weeks. Error bars indicate s.d. (d) Liver, spleen, and kidney histology. Livers, spleens, and kidneys were harvested from mice before, 1 day, and 4 weeks after intravenous injection of LPSiNP (20 mg/kg). Organs were stained with hematoxylin and eosin. The arrows indicate the LPSiNP taken up by macrophages in the liver. The scale bar is 50 μm for all images.

FIGS. 3A-G shows in vitro, in vivo and ex vivo fluorescence imaging with luminescent porous Si nanoparticles (LPSiNP). (a) In vitro cellular imaging with LPSiNP. HeLa cells were treated with LPSiNP for 2 h and then imaged. Red and blue indicate LPSiNP and cell nuclei, respectively. The scale bar is 20 μm. (b) In vivo fluorescence image of LPSiNP (20 μl of 0.1 mg/mL) injected subcutaneously and intramuscularly on each flank of a mouse. (c) In vivo images of LPSiNP and dextran-coated LPSiNP (D-LPSiNP). The mice were imaged at multiple time points after intravenous injection of LPSiNP and D-LPSiNP (20 mg/kg). Arrowhead and arrow with solid line indicate liver and bladder, respectively. (d) In vivo image showing the clearance of a portion of the injected dose of LPSiNP into the bladder, 1 h post-injection. Li and Bl indicate liver and bladder, respectively. (e) Lateral image of the same mice shown in (c), 8 h after LPSiNP or D-LPSiNP injection. Arrows with dashed line indicate spleen. (f) Fluorescence images showing the ex vivo biodistribution of LPSiNP and D-LPSiNP in mouse. Organs were harvested from the animals shown in (c), 24 h after injection. Li, Sp, K, LN, H, Bl, Lu, Sk, and Br indicate liver, spleen, kidney, lymph nodes, heart, bladder, lung, skin, and brain, respectively. (g) Fluorescence histology images of livers and spleens from the mice shown in (c) and (f), 24 h after injection. Red and blue indicate (D-)LPSiNP and cell nuclei, respectively. The scale bar is 50 μm for all images.

FIGS. 4A-D shows fluorescence images of tumors containing dextran-coated luminescent porous Si nanoparticles (D-LPSiNP). (a) Fluorescence images of D-LPSiNP as a function of concentration using different excitation filters (GFP: 445-490 nm and 1 s exposure time, DsRed: 500-550 nm, 2 s exposure time, Cy5.5: 615-665 nm, 8 s exposure time, and ICG: 710-760 nm, 20 s exposure time). The emission filter used is ICG (810-875 nm). (b) Representative fluorescence images of mouse bearing an MDA-MB-435 tumor. The mouse was imaged using a Cy5.5 excitation filter and ICG emission filter at the indicated times after intravenous injection of D-LPSiNP (20 mg/kg). Note that a strong signal from D-LPSiNP is observed in the tumor, indicating significant passive accumulation in the tumor by the EPR effect. (c) Ex vivo fluorescence images of tumor and muscle around the tumor from the mouse used in (b). (d) Fluorescence images of a tumor slice from the mouse in (b). Red and blue indicate D-LPSiNP and cell nuclei (DAPI stain), respectively. The scale bar is 100 μm.

FIG. 5 shows a schematic diagram of the synthesis of luminescent porous silicon nanoparticles (LPSiNP). A porous silicon layer with a pore size range of 5-10 nm is first etched into the single-crystal silicon substrate in ethanolic HF solution. The entire porous nanostructure is removed from the Si substrate by application of a current pulse. The freestanding hydrogen-terminated porous silicon film is then placed in an aqueous solution and fractured into multi-sized particles by overnight ultrasonication. The particles are then filtered through a 0.22 μm porous filtration membrane to obtain the porous silicon nanoparticles. Finally, the nanoparticles are incubated in an aqueous solution to activate their luminescence.

FIG. 6 shows FTIR spectra of porous silicon film and luminescent porous silicon nanoparticles (LPSiNP) shown in FIG. 5. The hydrogen-terminated surface of the as-etched porous silicon film is converted to silicon dioxide in the porous silicon nanoparticles. The oxide layer passivates the nanoparticle surface and also generates interfacial oxides, giving rise to a strong NIR photoluminescence.

FIG. 7 shows a photoluminescence spectra of LPSiNP, acquired during activation in deionized (DI) water at room temperature (1 d indicates 1 day after immersion in DI water). Note the increase in PL intensity and slight blue-shifting of the peak maximum with time.

FIG. 8A-E show characterization of three types of LPSiNP prepared with different porous nanostructures. (a) Analysis of porous nanostructures of LPSiNP by SEM and gas adsorption (BET/BJH) methods. The particle size values (by Dynamic Light Scattering, DLS) are the means plus/minus one standard deviation for three batches of LPSiNP, and the pore size values (by SEM) are averages of >10 different pores from randomly selected LPSiNP. (b) Pore size distributions and pore volume in LPSiNP determined by gas adsorption (BJH and dV/dw methods). (c) Photoluminescence spectra of LPSiNP (λₑₓ=370 nm). (d) In vitro degradation of LPSiNP in PBS solution at 37° C. as a function of time. Note that LPSiNP prepared using the etching condition with a current density of 50 mA/cm² show slightly slower degradation relative to the other two preparations of LPSiNP, suggesting that the their lower porosity may be responsible for the slower degradation. (e) Cytotoxicity of LPSiNP by calcein assay. HeLa cells were incubated with LPSiNP for 48 h and then viability was evaluated using the fluorogenic intracellular esterase sensor calcein acetoxymethylester (Calcein AM).

FIG. 9A-C shows characterization of LPSiNP with different average particle sizes, prepared using the same etching conditions (200 mA/cm² for 150 sec). The suffixes “-S” “-M” and “-L” refer to small (15 nm), medium (126 nm), and large (270 nm) particles. (a) Effect of nanoparticle size on the blood circulation half-life in mouse (n=3). Note that the LPSiNP-M show the longest circulation times relative to LPSiNP with other sizes. The LPSiNP-S are cleared rapidly by the kidney due to their small size (close to the typical renal clearance range of <5.5 nm) while the LPSiNP-L are cleared rapidly by the spleen or lung non-specifically due to their large size. The size and zeta potential values obtained by DLS are the means plus/minus one standard deviation for three lots of LPSiNP. The blood half-life values were obtained by fitting the concentration of silicon in each blood sample at each time point to a single-exponential equation using a one-compartment open pharmacokinetic model. (b) Photoluminescence spectra of the LPSiNP with different sizes (λₑₓ=370 nm). (c) Cytotoxicity of the LPSiNP with different sizes by Calcein assay. HeLa cells were incubated with LPSiNP for 48 h and then their viability was evaluated using the fluorogenic intracellular esterase sensor Calcein acetoxymethylester (Calcein AM).

FIG. 10 demonstrates changes in fluorescence intensity of LPSiNP dispersed in aqueous solution exposed to air during continuous exposure to a 100 W mercury lamp, compared with organic dyes commonly used in biological imaging experiments (Cy5.5, Cy7, and fluorescein). Excitation wavelengths of 355±25 nm for LPSiNP, 480±20 nm for fluorescein, 650±22 nm for Cy5.5, and 710±35 nm for Cy7, and emission wavelengths of 435 nm (long pass) for LPSiNP, 535±25 nm for fluorescein, 710±25 nm for Cy5.5, and 800±35 nm for Cy7 were used for these experiments. The fluorescence intensities were monitored with a thermoelectrically cooled CCD camera.

FIG. 11 shows quantum yield (QY=10.2%) of luminescent porous Si nanoparticles (LPSiNP) compared to Rhodamine 101 (QY=100%).

FIG. 12 shows evolution of photoluminescence spectrum of LPSiNP during degradation under physiological conditions (in PBS at 37° C.). The maximum intensity of the photoluminescence spectrum at each time point was used for FIG. 1d.

FIG. 13A-D shows characterization of DOX-loaded LPSiNP (DOX-LPSiNP). Zeta potential of LPSiNP increases from −52 mV to −39.1 mV after DOX loading. (a) Appearance of silicon in solution (by ICP-OES) from a sample of LPSiNP and DOX-LPSiNP incubated in PBS solution at 37° C. as a function of time. (b) Blood concentration of silicon (by ICP-OES) or DOX (by fluorescence) for mice injected with LPSiNP, DOX-LPSiNP, or free DOX as a function of time (n=3). DOX in DOX-LPSiNP were able to circulate for a longer period of time than free DOX by incorporating DOX in the porous nanostructure of LPSiNP. Note that there is no significant difference in the circulation times between DOX-LPSiNP and LPSiNP. (c) Biodistribution of DOX from mice (n=3) injected with free DOX or DOX-LPSiNP. The tissues were homogenized 24 h after single intravenous injection of free DOX or DOX-LPSiNP (concentration of DOX in all experiments was 2 mgDOX/kg, corresponding to 45.5 mg/kg of LPSiNP in the DOX-LPSiNP formulation) and the DOX fluorescence (λₑₓ=480 nm and λₑₘ=590 nm) in each tissue was analyzed as % injection dose (% ID) per gram of wet tissue. The results show that biodistribution of DOX from DOX-LPSiNP is similar to that of LPSiNP as shown in FIG. 2b, confirming that LPSiNP retain the loaded DOX during the circulation. In contrast, a significant amount of free DOX accumulated in kidney, lung, and heart, which may induce in vivo acute or chronic toxicity (*p<0.05). (d) Body mass change in mice injected with free DOX, and DOX-LPSiNP (concentration of DOX in all experiments was 2 mgDOX/kg, corresponding to 45.5 mg/kg of LPSiNP in the DOX-LPSiNP formulation) compared with PBS control (n=3). There was no statistically significant difference in the weight change between control (PBS), free DOX, and, DOX-LPSiNP over a period of 3 weeks.

FIG. 14A-D are optical microscope images of HeLa cells incubated with LPSiNP at a concentration of (a) 0 mg/mL, (b) 0.013 mg/mL, (c) 0.05 mg/mL, and (d) 0.2 mg/mL. The cells were rinsed three times using cell medium (no phenol red) 48 h after incubation and immediately imaged using an inverted optical microscope. The scale bar is 20 μm.

FIG. 15A-C shows in vitro cellular imaging with LPSiNP. HeLa cells were treated with LPSiNP for 2 h, fixed and then imaged. (a) Fluorescence microscope images of cellular uptake and binding of LPSiNP. The nanoparticles can be imaged in vitro under both excitation wavelengths indicated on the left side of the images (λₑₘ=650 nm long pass). (b) Confocal fluorescence microscope images of cellular uptake of LPSiNP (λₑₓ=488 nm and λₑₘ=650 nm long pass). (c) Multi-photon fluorescence microscope image of celluar uptake of LPSiNP (λₑₓ=750 nm). The LPSiNP are clearly observable inside the cells under two-photon excitation conditions as well as with single-photon excitation, in agreement with previous observations with porous silicon chips. The scale bar is 20 μm.

FIG. 16A-D show characterization of dextran-coated luminescent porous silicon nanoparticles (D-LPSiNP). (a) Dynamic light scattering size data obtained from LPSiNP and D-LPSiNP. Note that the size and zeta potential increases from 125 nm to 151 nm and from −52 mV to −43.5 mV after dextran coating, respectively. Although these samples display a relatively large size distribution, there are several FDA (US Food and Drug Administration)-approved nanoparticle formulations that span this range, such as liposomal doxorubicin (Doxil®), albumin-bound Paclitaxel (Abraxane®), and dextran-coated iron oxide nanoparticles (Feridex®). (b) Photoluminescence spectra of LPSiNP and D-LPSiNP with UV excitation (λ=370 nm). (c) In vitro degradation of LPSiNP and D-LPSiNP (in PBS at 37° C.). Degradation is monitored as the intensity of photoluminescence from the nanoparticle. (d) Blood concentration of silicon (by ICP-OES) for mice (n=3) injected with LPSiNP or D-LPSiNP as a function of time. LPSiNP are cleared in a short time (blood half-life: 27.6±1.8 min) while D-LPSiNP circulate for a longer period of time (blood half-life: 82.0±15.8 min).

FIG. 17 shows fluorescence images of mouse bearing MDA-MB-435 tumor after injection of dextran-coated luminescent porous silicon nanoparticles (D-LPSiNP) using different excitation filters (GFP: 445-490 nm, DsRed: 500-550 nm, Cy5.5: 615-665 nm, and 20 s exposure time for all images). The mouse was imaged 8 h after intravenous injection of D-LPSiNP (20 mg/kg). The emission filter used is ICG (810-875 nm).

### DETAILED DESCRIPTION

As used herein and in the appended claims, the singular forms “a,” “and,” and “the” include plural referents unless the context clearly dictates otherwise. Thus, for example, reference to “a pore” includes a plurality of such pores and reference to “the drug” includes reference to one or more drugs known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

A desirable design criterion for improving the biocompatibility of nanomaterials would involve the incorporation of controllable rates of self-destruction, through which components could be hierarchically degraded into harmless, renally-cleared products after performing their in vivo function.

Electrochemically etched porous silicon has exhibited considerable potential for biological applications due to its biocompatibility, biodegradability, encoding property for multiplexed detection, and tunable porous nanostructure for drug delivery. For in vivo use, silicon nanoparticles provide attractive chemical alternatives to heavy metal-containing quantum dots (QDs), which have been shown to be toxic in biological environments.

Silicon is the chemical element that has the symbol Si and atomic number 14. Silicon occasionally occurs as the pure free element in nature, but is more widely distributed as various forms of silicon dioxide (silica) or silicates. Silicon is used in the electronics industry where substantially pure and highly pure silicon are used for the formation of wafers. Pure silicon is used to produce ultra-pure silicon wafers used in the semiconductor industry, in electronics and in photovoltaic applications. Ultra-pure silicon can be doped with other elements to adjust its electrical response by controlling the number and charge (positive or negative) of current carriers. Such control is desirable for transistors, solar cells, integrated circuits, microprocessors, semiconductor detectors and other semiconductor devices which are used in electronics and other high-tech applications. In photonics, silicon can be used as a continuous wave Raman laser medium to produce coherent light. Hydrogenated amorphous silicon is used in the production of low-cost, large-area electronics in applications such as LCDs, and of large-area, low-cost thin-film solar cells. Accordingly, most commonly purchased silicon is in the form of silicon wafers. Silicon when metabolized by the body is converted to silane, a compound that when accumulated has toxic effects.

Silicon oxide typically refers to a silicon element linked to a single reactive oxygen species (e.g., a radical). Such silicon oxide compounds are useful for the addition of carbon or other desirable elements wherein a bond is formed between the reactive oxygen and the desired element or chemical side chain. Silicon oxides are useful for the formation of hydrogenated silicon oxycarbide (H:SiOC) films having low dielectric constant and a light transmittance. Such Si—O—X (wherein X is any suitable element other than oxygen) compounds are formed using complex reactions including reacting a methyl-containing silane in a controlled oxygen environment using plasma enhanced or ozone assisted chemical vapor deposition to produce the films.

Silicon dioxide refers to the compound SiO₂ (sometime referred to as silica). Silicon dioxide is formed when silicon is exposed to oxygen (or air). A thin layer (approximately 1 nm or 10 Å) of so-called ‘native oxide’ is formed on the surface when silicon is exposed to air under ambient conditions. Higher temperatures and alternate environments are used to grow layers of silicon dioxide on silicon. Silicon dioxide is inert and harmless. When silica is ingested orally, it passes unchanged through the gastrointestinal tract, exiting in the feces, leaving no trace behind. Small pieces of silicon dioxide are equally harmless, so long as they are not large enough to mechanically obstruct the GI tract or fluid flow, or jagged enough to lacerate the GI lining, vessel or other tissue. Silicon dioxide produces no fumes and is insoluble in vivo. It is indigestible, with zero nutritional value and zero toxicity. Silicon dioxide has covalent bonding and forms a network structure. Hydrofluoric acid (HF) is used to remove or pattern silicon dioxide in the semiconductor industry.

Silicon is an essential trace element that is linked to the health of bone and connective tissues. The chemical species of relevance to the toxicity of porous Si are silane (SiH₄) and dissolved oxides of silicon; three important chemical reactions of these species are given in Eq. (1)-(3). The surface of porous Si contains Si—H, SiH₂, and SiH₃ species that can readily convert to silane. Silane is chemically reactive (Eq. (1)) and toxic, especially upon inhalation. Like silane, the native SiHₓ species on the porous Si surface readily oxidize in aqueous media. Silicon itself is thermodynamically unstable towards oxidation, and even water has sufficient oxidizing potential to make this reaction spontaneous Eq. (2). The passivating action of SiO₂ and Si—H (for samples immersed in HF solutions) make the spontaneous aqueous dissolution of Si kinetically slow. Because of its highly porous nanostructure, oxidized porous Si can release relatively large amounts of silicon-containing species into solution in a short time. The soluble forms of SiO₂ exist as various silicic acid compounds with the orthosilicate (SiO₄⁴⁻) ion on as the basic building block (Eq. (3)), and these oxides can be toxic in high doses. Because the body can handle and eliminate silicic acid, the important issue with porous Si-based drug delivery systems is the rate at which they degrade and resorb.

SiH₄+2H₂O→SiO₂+4H₂ (1)

Si+O₂→SiO₂ (2)

SiO₂+2H₂O→Si(OH)₄ (3)

Surface chemistry plays a large role in controlling the degradation properties of porous Si in vivo. After Si is electrochemically etched, the surface is covered with reactive hydride species. These chemical functionalities provide a versatile starting point for various reactions that determine the dissolution rates in aqueous media, allow the attachment of homing species, and control the release rates of drugs. The two most important modification reactions are chemical oxidation (Eq. (2)) and grafting of Si—C species.

The various embodiments provided herein are generally directed to systems and methods for producing a drug delivery device that can deliver time dependent dosing without the need for electronics or power as well as imaging agents for imaging and diagnosis of various diseases or disorders including cancers, tumors and other cell proliferative diseases and disorders, inflammatory diseases and disorders and tissue damage. Accordingly, the disclosure recognizes and addresses an important and unmet medical need for a minimally invasive, controllable and monitorable drug delivery system and methods of using the system that would enable long acting local treatment of both extraocular and intraocular diseases as well as tissue imaging. Such luminescent materials are useful for monitoring in vivo conditions or biological changes in cells or environments where toxicity is an issue.

The disclosure provides luminescent porous smart dust (i.e., luminescent porous silicon nanoparticles—LPSiNPs). The LPSiNP materials can be generated by first producing a silicon layer with a pore size range of 2-100 nm (e.g., 5-10 nm, 10-20 nm, 20-30 nm etc.). The silicon layer is etched into the single-crystal silicon substrate in ethanolic HF solution, as described more fully below. The entire porous nanostructure is removed from the Si substrate by application of a current pulse. The freestanding hydrogen-terminated porous silicon film is then placed in an aqueous solution and fractured into multi-sized particles by, for example, overnight ultrasonication. The particles can then be filtered if desired (e.g., through a 0.22 μm porous filtration membrane or other size separating device) to obtain porous silicon nanoparticles. For example, separation or size control of LPSiNPs can be achieved by passing the colloidal suspension through physical filters, by centrifugation of the suspension, by electrophoresis, by size exclusion chromatography, or by electrostatic precipitation. The nanoparticles are incubated in an aqueous oxidizing solution to activate their luminescence.

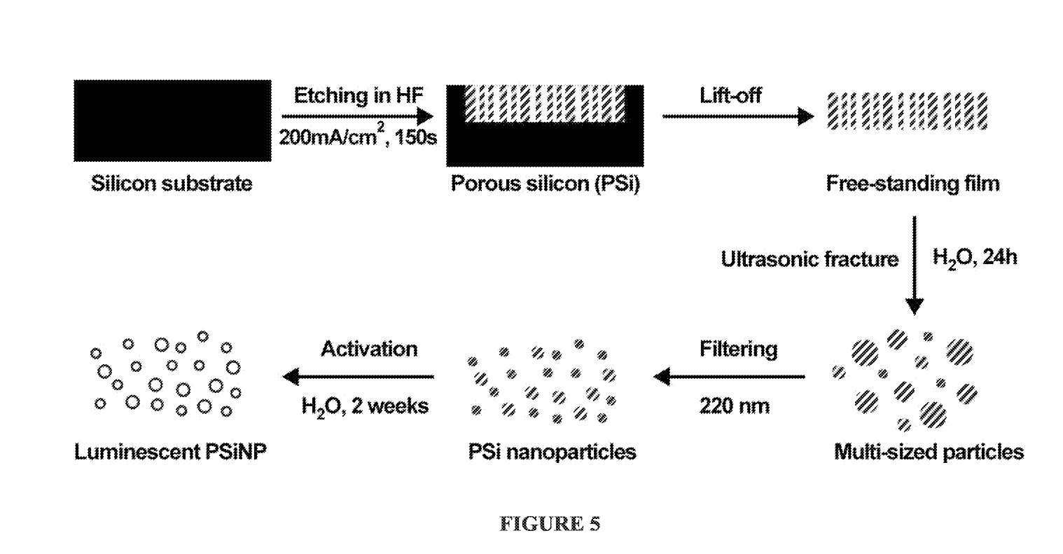

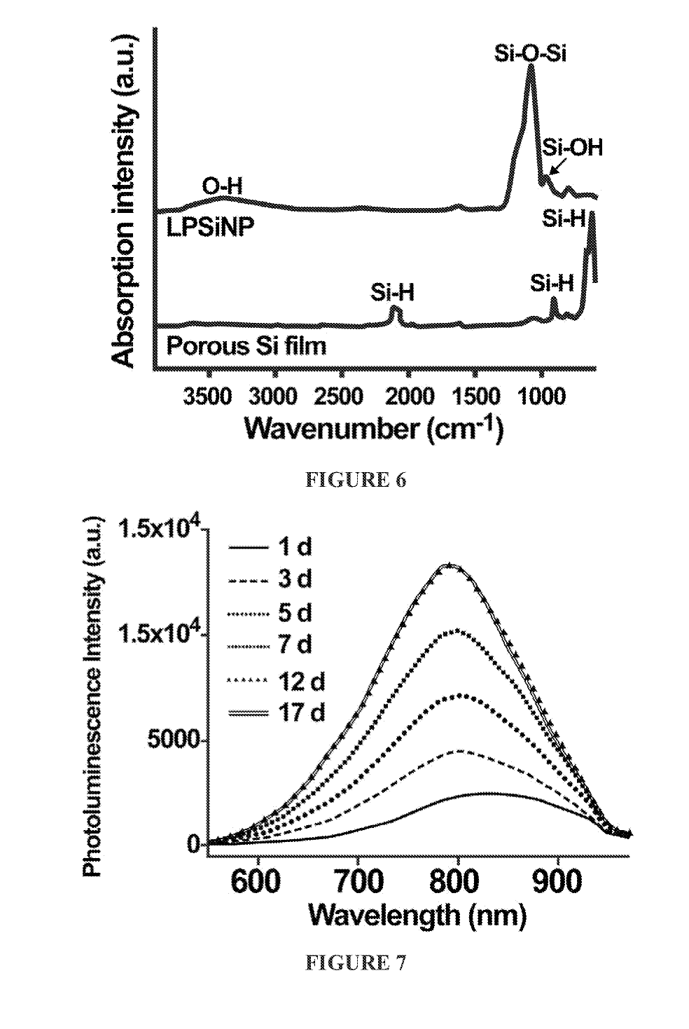

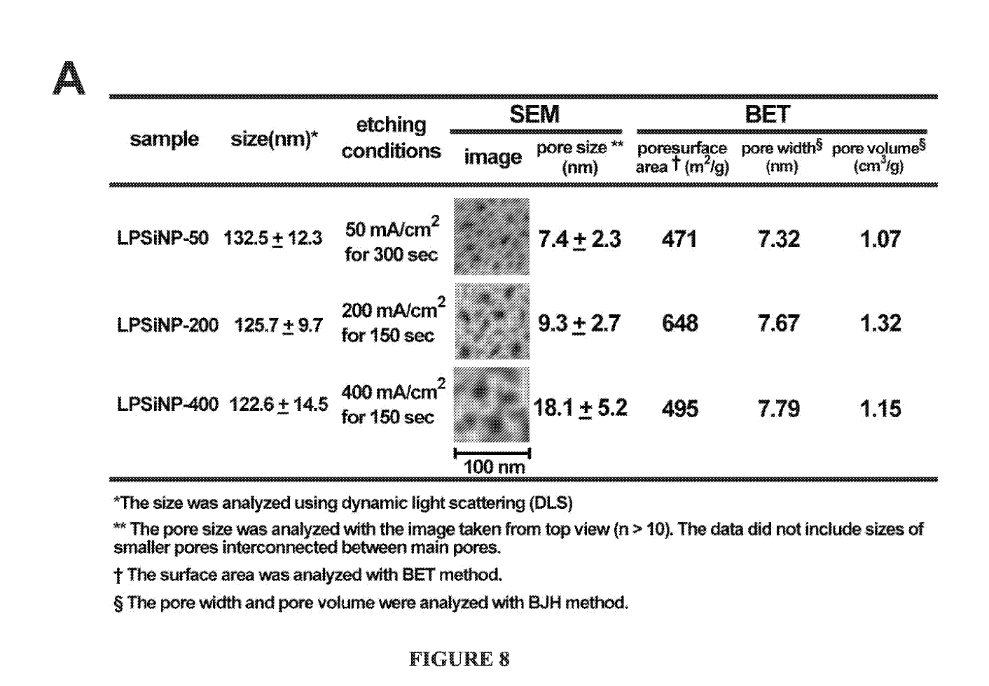

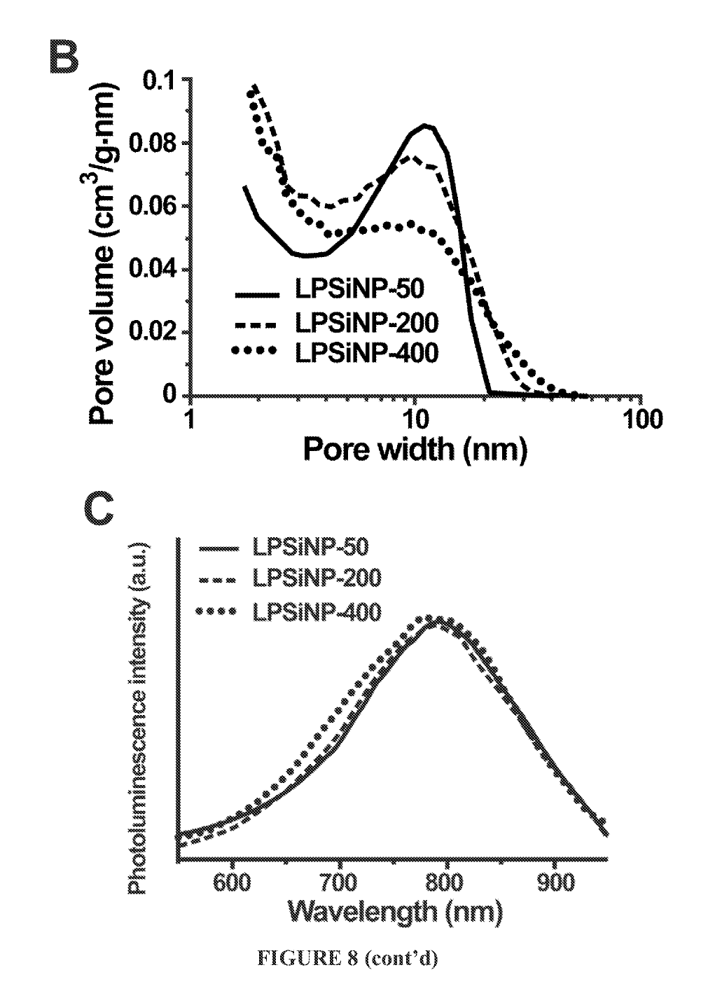

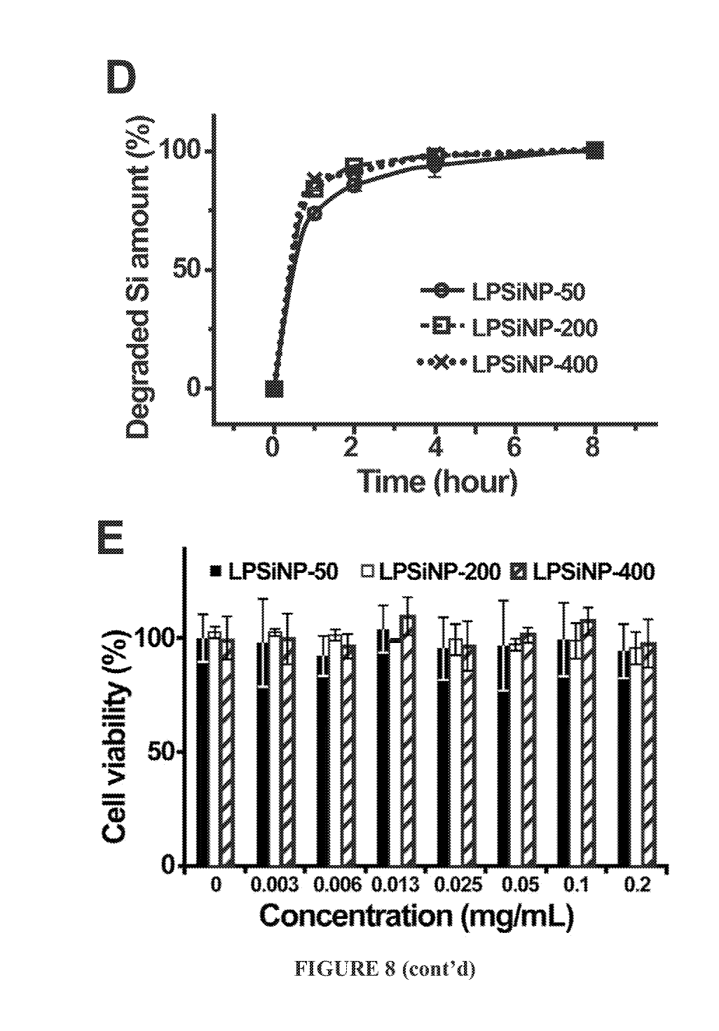

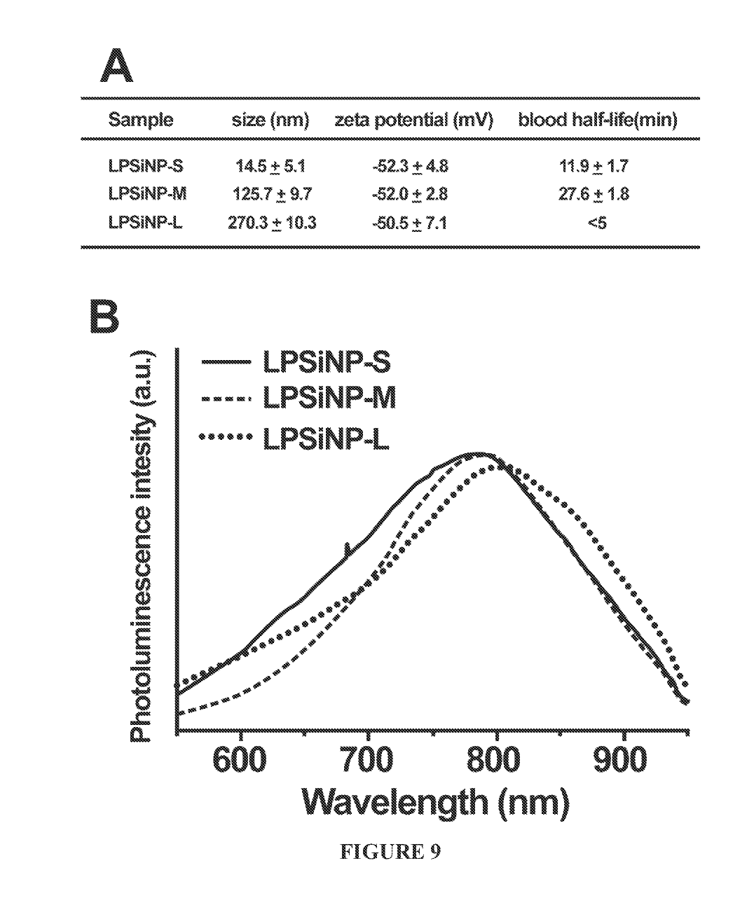

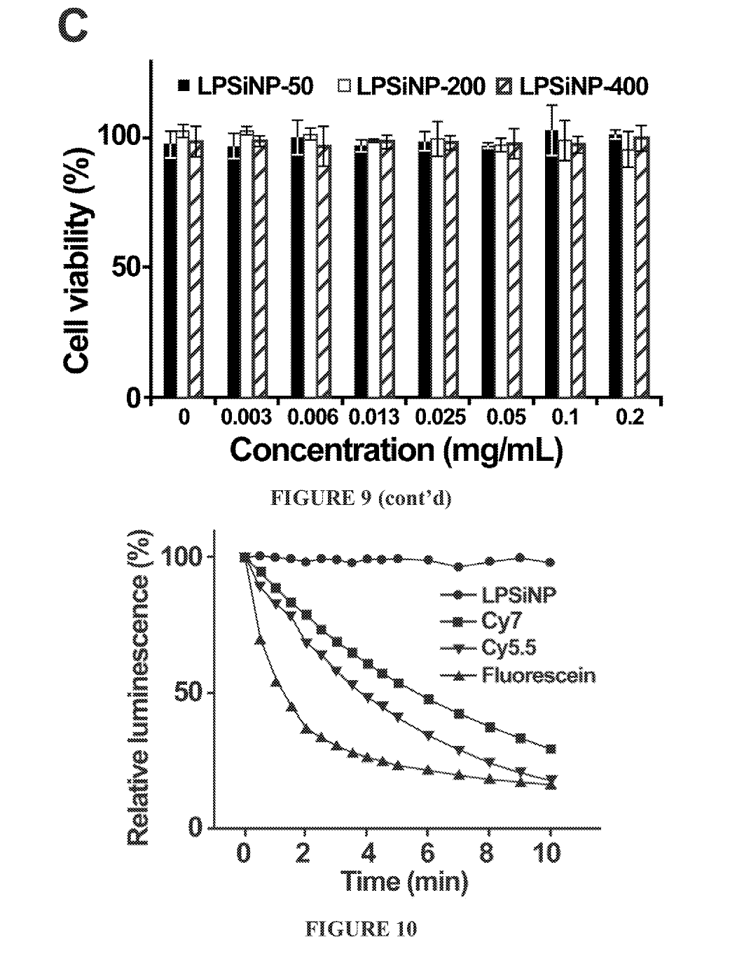

The activation of luminescence is performed in an aqueous solution (see, e.g., FIG. 5). During the activation silicon oxide grows on the hydrogen-terminated porous silicon surface, generating significant luminescence attributed to quantum confinement effects and to defects localized at the Si/SiO₂ interface (see, e.g., FIGS. 5 and 6). The preparation conditions of the nanoparticles can be optimized to provide pore volumes and surface areas suitable for loading of therapeutics and for desired in vivo circulation times while maintaining an acceptable degradation rate (FIG. 8-9).

Various aqueous buffers that are oxidizing (or neutral to basic) can be used. In some embodiments, an aqueous buffer selected from the group consisting of an aqueous borate buffer, a phosphate buffered saline, and sodium hydroxide. For example, in one embodiment, a borate aqueous buffer is useful. Borates in chemistry are chemical compounds containing boron oxoanions, with boron in oxidation state +3. The simplest borate ion is the trigonal planar, BO₃³⁻, although many others are known. In aqueous solution borate exists in many forms. In acid and near-neutral conditions, it is boric acid, commonly written as H₃BO₃ but more correctly B(OH)₃. The pKa of boric acid is 9.14 at 25° C. Boric acid does not dissociate in aqueous solution, but is acidic due to its interaction with water molecules, forming tetrahydroxyborate.

In one embodiment, the disclosure provides a method of generating a luminescent porous Si nanoparticles (LPSiNP). The method comprises electrochemical etching of a p-type silicon wafer by application of a constant current density of about 200 mA/cm² in an aqueous HF/ethanol electrolyte. The resulting freestanding film of porous silicon nanostructure is then removed from the crystalline silicon substrate by application of a current pulse of about 4 mA/cm² in an aqueous HF/ethanol electrolyte. The freestanding hydrogen-terminated porous silicon film is subsequently fractured, e.g,. by sonication, and then filtered to obtain a desired particle size (e.g., through a 0.22 μm filtration membrane (Millipore)). Other methods of size selecting the nanoparticles can be performed by centrifugation and chromatography. The nanoparticles are further incubated in deionized (DI) water or other oxidizing aqueous environment such as, for example, a borate aqueous buffer, to activate their luminescence (e.g., in one embodiment in the near-infrared range). The resulting LPSiNP can then be further modified or loaded with a desired drug agent or other factor. For example, for dextran-coated LPSiNP (D-LPSiNP), dextran (MW˜20,000, Sigma) is physically absorbed on LPSiNP. Various methods off attaching a coating are known. For example, the process for coating LPSiNPs can be one of, or a combination of processes including physical adsorption, physical absorption, covalent attachment, electrostatic adsorption, precipitation of an insoluble overcoating, electroplating, or electroless plating.

Following is further detail for the production of the LPSiNPs of the disclosure. Photonic crystals are produced from porous silicon and porous silicon/polymer composites, or porous Si film or polymer replica or Si-polymer composite may be generated as a sheet for an exoplant. Pulsed electrochemical etching of a silicon chip produces a multilayered porous nanostructure. A convenient feature of porous Si is that the average pore size can be controlled over a wide range by appropriate choice of current, HF concentration, wafer resistivity, and electrode configuration used in the electrochemical etch. This tunability of the pore dimensions, porosity, and surface area is especially advantageous.

The thickness, pore size, and porosity of a given film is controlled by the current density, duration of the etch cycle, and etchant solution composition. In addition, a porous silicon film can be used as a template to generate an imprint of biologically compatible or bioresorbable materials. The porous silicon film or its imprint possess a sinusoidally varying porosity gradient, providing sharp features in the optical reflectivity spectrum that can be used to monitor the presence or absence of chemicals trapped in the pores. It has been shown that the particles (“smart dust”) made from the porous silicon films by mechanical grinding or by ultrasonic fracture still carry the optical reflectivity spectrum.

Porous Si is a product of an electrochemical anodization of single crystalline Si wafers in a hydrofluoric acid electrolyte solution. Pore morphology and pore size can be varied by controlling the current density, the type and concentration of dopant, the crystalline orientation of the wafer, and the electrolyte concentration in order to form macro-, meso-, and micropores. Pore sizes ranging from 1 nm to a few microns can be prepared. The type of dopant in the original silicon wafer is important because it determines the availability of valence band holes that are the key oxidizing equivalents in the reaction shown in FIG. 5. In general the relationships of dopant to morphology can be segregated into four groups based on the type and concentration of the dopant: n-type, p-type, highly doped n-type, and highly doped p-type. By “highly doped,” is meant dopant levels at which the conductivity behavior of the material is more metallic than semiconducting. For n-type silicon wafers with a relatively moderate doping level, exclusion of valence band holes from the space charge region determines the pore diameter. Quantum confinement effects are thought to limit pore size in moderately p-doped material. For both dopant types the reaction is crystal face selective, with the pores propagating primarily in the direction of the single crystal. For example, electrochemically driven reactions use an electrolyte containing hydrofluoric acid. Application of anodic current oxidizes a surface silicon atom, which is then attacked by fluoride. The net process is a 4 electron oxidation, but only two equivalents are supplied by the current source. The other two equivalents come from reduction of protons in the solution by surface SiF₂ species. Pore formation occurs as Si atoms are removed in the form of SiF₄, which reacts with two equivalents of F⁻ in solution to form SiF₆²⁻.

The porosity of a growing porous Si layer is proportional to the current density being applied, and it typically ranges between 40 and 80%. Pores form at the Si/porous Si interface, and once formed, the morphology of the pores does not change significantly for the remainder of the etching process. However, the porosity of a growing layer can be altered by changing the applied current. The film will continue to grow with this new porosity until the current changes.

This feature allows the construction of layered nanostructures simply by modulating the applied current during an etch. For example, one dimensional photonic crystals consisting of a stack of layers with alternating refractive index can be prepared by periodically modulating the current during an etch.

Stain etching is an alternative to the electrochemical method for fabrication of porous Si powders. The term stain etching refers to the brownish or reddish color of the film of porous Si that is generated on a crystalline silicon material subjected to the process. In the stain etching procedure, a chemical oxidant (typically nitric acid) replaces the power supply used in the electrochemically driven reaction. HF is typically used as an ingredient, and various other additives are used to control the reaction. Stain etching generally is less reproducible than the electrochemical process, although recent advances have improved the reliability of the process substantially. Porous Si powders prepared by stain etch are commercially available.

For in vivo applications, it is often desirable to prepare porous Si in the form of particles. The porous layer can be removed from the Si substrate with a procedure commonly referred to as “electropolishing” or “lift-off.” The etching electrolyte is replaced with one containing a lower concentration of HF and a current pulse is applied for several seconds. The lower concentration of HF results in a diffusion limited situation that removes silicon from the crystalline Si/porous Si interface faster than pores can propagate. The result is an undercutting of the porous layer, releasing it from the Si substrate. The freestanding porous Si film can then be removed with tweezers or a vigorous rinse. The film can then be converted into microparticles by ultrasonic fracture. Conventional lithography or microdroplet patterning methods can also be used if particles with more uniform shapes are desired.

The ability to easily tune the pore sizes and volumes during the electrochemical etch is a unique property of porous Si that is very useful for drug delivery applications. Other porous materials generally require a more complicated design protocol to control pore size, and even then, the available pore sizes tend to span a limited range. With electrochemically prepared porous Si, control over porosity and pore size is obtained by adjusting the current settings during etching. Typically, larger current density produces larger pores. Large pores are desirable when incorporating sizable molecules or drugs within the pores. Pore size and porosity is important not only for drug loading; it also determines degradation rates of the porous Si host matrix.

Smaller pores provide more surface area and expose more sites for attack of aqueous media. The smaller porous filaments within the film yield greater dissolution rates, providing a convenient means to control degradation rates of the porous Si host.

The fractionated mixture can be filtered, centrifuged, column sized to obtain a desired nanostructure size. For example, as depicted in FIG. 5, a filter is used to obtain nanostructures smaller than 220 nm.

With its high surface area, porous Si is particularly susceptible to air or water oxidation. Once oxidized, nanophase SiO₂ readily dissolves in aqueous media, and surfactants or nucleophiles accelerate the process. Si—O bonds are easy to prepare on porous Si by oxidation, and a variety of chemical or electrochemical oxidants can be used. Thermal oxidation in air tends to produce a relatively stable oxide, in particular if the reaction is performed at >600° C. Ozone oxidation, usually performed at room temperature, forms a more hydrated oxide that dissolves quickly in aqueous media.

Milder chemical oxidants, such as dimethyl sulfoxide (DMSO, Eq. (4)), benzoquenone, or pyridine, can also be used for this reaction. Mild oxidants are sometimes preferred because they can improve the mechanical stability of highly porous Si films, which are typically quite fragile.

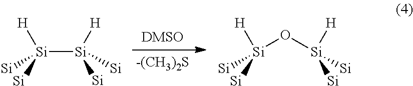

The mechanical instability of porous Si is directly related to the strain that is induced in the film as it is produced in the electrochemical etching process, and the volume expansion that accompanies thermal oxidation can also introduce strain. Mild chemical oxidants presumably attack porous Si preferentially at Si—Si bonds that are the most strained, and hence most reactive. As an alternative, nitrate is a stronger oxidant, and nitric acid solutions are used extensively in the preparation of porous Si particles from silicon powders by chemical stain etching.

Slow oxidation of the porous Si surface by dimethyl sulfoxide (DMSO), when coupled with dissolution of the newly formed oxide by HF, is a mild means to enlarge the pores in porous Si films. Aqueous solutions of bases such as KOH can also be used to enlarge the pores after etching. Electrochemical oxidation, in which a porous Si sample is anodized in the presence of a mineral acid such as H₂SO₄, yields a fairly stable oxide. Oxidation imparts hydrophilicity to the porous structure, enabling the incorporation and adsorption of hydrophilic drugs or biomolecules within the pores. Aqueous oxidation in the presence of various ions including Ca²⁺ generates a calicified form of porous Si that has been shown to be bioactive and is of particular interest for in vivo applications. Calcification can be enhanced by application of a DC electric current.

Carbon grafting stabilizes porous Si against dissolution in aqueous media, but the surface must still avoid the non-specific binding of proteins and other species that can lead to opsonization or encapsulation. Reactions that place a polyethylene glycol (PEG) linker on a porous Si surface have been employed to this end. A short-chain PEG linker yields a hydrophilic surface that is capable of passing biomolecules into or out of the pores without binding them strongly. The distal end of the PEG linker can be modified to allow coupling of other species, such as drugs, cleavable linkers, or targeting moieties, to the material.

The oxides of porous Si are easy to functionalize using conventional silanol chemistries. When small pores are present (as with p-type samples), monoalkoxydimethylsilanes (RC—Si(Me)₂—R′) can be more effective than trialkoxysilanes ((RO)₃Si—R′) as surface linkers. This is because trialkoxysilanes oligomerize and clog smaller pore openings, especially when the reagent is used at higher concentrations.

Whereas Si—C chemistries are robust and versatile, chemistries involving Si—O bonds represent an attractive alternative for at least two reasons. First, the timescale in which highly porous SiO₂ is stable in aqueous media is consistent with many short-term drug delivery applications—typically 20 min to a few hours. Second, a porous SiO₂ sample that contains no additional stabilizing chemistries is less likely to produce toxic or antigenic side effects. If it is desired that the porous Si material be stable in vivo for long periods (for example, an extended release formulation or an in vivo biosensor), Si—C chemistries such as hydrosilylation with end capping or thermal carbonization with acetylene is useful. If a longer-lived oxide matrix is desired, silicon oxides formed at higher temperatures (>700° C.) are significantly more stable in aqueous media than those formed at lower temperatures or by ozone oxidation.

Again, once the smart dust is produced and filtered to a desired size, aqueous oxidation imparts a desired luminescence.

The LPSiNPs of the disclosure provide a device and method for drug delivery and tissue and disease (e.g., tumor) monitoring. For example, the LPSiNPs of the disclosure provide a device and method for intravitreal drug delivery that promotes sustained intraocular therapeutic drug levels with minimal invasiveness and elimination of systemic side effects. Impregnation of the porous material may proceed in several ways. The disclosure also provide methods for targeted delivery and analysis of the location of a drug-delivery LPSiNP device of the disclosure.

A drug-delivery LPSiNP device can include any number of candidate drugs depending upon the type of condition, tissue, cancer to be treated. A candidate drug may be “physically” trapped within the pores, or, the pores themselves may be chemically modified to bind the candidate drug.

More specifically, “physical trapping” is similar to building a ship in a bottle, where the “ship” is the candidate drug and the “bottle” is the nanometer-scale pores in the porous Si matrix. Small molecules can be trapped in the porous matrix by oxidizing the porous Si around the molecule. Since oxidation of silicon adds two atoms of oxygen per atom of Si to the material, there is a significant increase in volume of the matrix upon oxidation. This has the effect of swelling the pore walls and shrinking the free volume inside the pores, and under the appropriate conditions, molecules present in the pores during oxidation become trapped in the oxide matrix. One aspect of the trapping process is the increased concentration of the active ingredient which occurs during the trapping process. The crystals may present a negatively charged environment and an active ingredient, such as proteins and other drugs, may be concentrated in the crystals to levels much higher than the free concentration of the active ingredient in solution. This can result in 10 to 100 fold or more increase in active ingredient concentration when associated with a crystal. The oxidizing can be performed at repeated intervals by performing layered oxidation. For example, a biological agent or drug can be trapped in the pores by controlled addition of oxidants. Oxidation of the freshly prepared (hydride-terminated) porous Si material results in an effective shrinking of the pores. This occurs because the silicon oxide formed has a larger volume than the Si starting material. If a drug is also present in the solution that contains the oxidant, the drug becomes trapped in the pores.

Furthermore the porous silicon oxide can comprise a higher concentration of a biological agent or drug than a non-oxidized Si hydride material.

The free volume in a porous Si film is typically between 50 and 80%. Oxidation should reduce this value somewhat, but the free volume is expected to remain quite high. Most of the current drug delivery materials are dense solids and can deliver a small percentage of drug by weight. The amount of drug that can be loaded into the porous Si material is expected to be much larger than, for example, surface-modified nanoparticles or polylactide (PLA) polymers.

Various approaches to load a molecular payload into a porous Si host have been explored, and they can be grouped into the following general categories: covalent attachment, physical trapping, and adsorption.

Covalent attachment provides a convenient means to link a biomolecular capture probe to the inner pore walls of porous Si for biosensor applications, and this approach can also be used to attach drug molecules. As described elsewhere herein, linking a biomolecule via Si—C bonds tends to be a more stable route than using Si—O bonds due to the susceptibility of the Si—O species to nucleophilic attack.

One of the more common approaches is to graft an organic molecule that contains a carboxyl species on the distal end of a terminal alkene. The alkene end participates in the hydrosilylation reaction, bonding to the Si surface and leaving the carboxy-terminus free for further chemical modification. A favorite linker molecule is undecylenic acid, which provides a hydrophobic 10 carbon aliphatic chain to insulate the linker from the porous Si surface. The drug payload can be attached directly to the carboxy group of the alkene, or it can be further separated from the surface with a PEG linker. Due to the stability of the Si—C bond, hydrosilylation is good way of attaching a payload to porous Si. The payload is only released when the covalent bonds are broken or the supporting porous Si matrix is degraded. For drug delivery this introduces a complication in that the drug may not release from the linker, resulting in a modified version of the drug being introduced into the body. In addition, a drug may be susceptible to attack by silane generated during the degradation of the porous Si scaffolding or by residual reactive species on the porous Si material itself.

Whereas covalent attachment and oxidative trapping approaches described above tend to trap their payloads fairly irreversibly, electrostatic adsorption represents essentially an ion exchange mechanism that holds molecules more weakly. Electrostatics is a useful means to affect more rapid drug delivery, as opposed to covalent or physical trapping approaches that release drug over a period of days, weeks, or months.

The affinity of a porous Si particle for a particular molecule can be controlled with surface chemistry. The surface of oxidized porous Si has a point of zero charge at a pH of around 2, and so it presents a negatively charged surface to most aqueous solutions of interest. At the appropriate pH, porous SiO2 spontaneously adsorbs positively charged proteins such as serum albumin, fibrinogen, protein A, immunoglobulin G (IgG), or horseradish peroxidase, concentrating them in the process.

Porous Si can also be made hydrophobic, and hydrophobic molecules such as the steroid dexamethasone or serum albumin can be loaded into these nanostructures. Hydrophilic molecules can also be loaded into such materials with the aid of the appropriate surfactant. The native hydride surface of porous Si is hydrophobic. Such techniques have been used for short-term loading and release. Because water is excluded from these hydrophobic surfaces, aqueous degradation and leaching reactions tend to be slow. The grafting of alkanes to the surface by hydrosilylation is commonly used to prepare materials that are stable in biological media; this stability derives in large part from the ability of the hydrophobic moieties to locally exclude water or dissolved nucleophiles.

While the disclosure provides for use with a virtually unlimited number of pharmaceutical candidates, several exemplary drugs will be discussed herein. For example, drug delivery for drugs used in treating ARMD and uveitis can be delivered using the LPSiNPs of the disclosure.

Other drugs or “active ingredient” that can be used with the smart dust of the disclosure include any one or any combination of the following, but are not limited to, anti-angiogenic compounds such as bevacizumab, ranibizumab, pegaptanib, and other compounds in the angiogenic cascade. Anti-cancer drugs such as, for example, chemotherapeutic compounds and/or derivatives thereof (e.g., 5-fluorouracil, vincristine, vinblastine, cisplatin, doxyrubicin, adriamycin, tamocifen, etc.). Also included are glucocorticosteroids such as dexamethasone, triamcinolone acetonide, fluocinolone acetonide and other comparable compounds in the corticosteroid and cortisene families. Also included are compounds such as antacids, anti-inflammatory substances, coronary dilators, cerebral dilators, peripheral vasodilators, anti-infectives, psychotropics, anti-manics, stimulants, anti-histamines, laxatives, decongestants, vitamins, gastrointestinal sedatives, anti-diarrheal preparations, anti-anginal drugs, vasodilators, anti-arrhythmics, anti-hypertensive drugs, vasoconstrictors and migraine treatments, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, bronchodilators, expectorants, cough suppressants, mucolytics, drugs affecting calcification and bone turnover and anti-uricemic drugs. Specific drugs include gastro-intestinal sedatives such as metoclopramide and propantheline bromide; antacids such as aluminum trisilicate, aluminum hydroxide, ranitidine and cimetidine; anti-inflammatory drugs such as phenylbutazone, indomethacin, naproxen, ibuprofen, flurbiprofen, diclofenac, dexamethasone, prednisone and prednisolone; coronary vasodilator drugs such as glyceryl trinitrate, isosorbide dinitrate and pentaerythritol tetranitrate; peripheral and cerebral vasodilators such as soloctidilum, vincamine, naftidrofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; anti-diarrheal drugs such as bisacodyl and magnesium hydroxide; the laxative drug, dioctyl sodium sulfosuccinate; nutritional supplements such as ascorbic acid, alpha tocopherol, thiamine and pyridoxine; anti-spasmodic drugs such as dicyclomine and diphenoxylate; drugs affecting the rhythm of the heart such as verapamil, nifedipine, diltiazem, procainamide, disopyramide, bretylium tosylate, quinidine sulfate and quinidine gluconate; drugs used in the treatment of hypertension such as propranolol hydrochloride, guanethidine monosulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid; anti-epileptic drugs such as phenytoin sodium and sodium valproate; neuromuscular drugs such as dantrolene sodium; substances used in the treatment of diabetes such as tolbutamide, disbenase glucagon and insulin; drugs used in the treatment of thyroid gland dysfunction such as triiodothyronine, thyroxine and propylthiouracil, diuretic drugs such as furosemide, chlorthalidone, hydrochlorthiazide, spironolactone and triamterene; the uterine relaxant drug ritodrine; appetite suppressants such as fenfluramine hydrochloride, phentermine and diethylproprion hydrochloride; anti-asthmatic and bronchodilator drugs such as aminophylline, theophylline, salbutamol, orciprenaline sulphate and terbutaline sulphate; expectorant drugs such as guaiphenesin; cough suppressants such as dextromethorphan and noscapine; mucolytic drugs such as carbocisteine; anti-septics such as cetylpyridinium chloride, tyrothricin and chlorhexidine; decongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; and calcification affecting agents such as biphosphonates, e.g., etidronate, pamidronate, alendronate, residronate, teludronate, clodronate and alondronate.

Insofar as the disclosure contemplates including a virtually unlimited number of drugs, in vitro pharmacokinetic studies can be used to determine the appropriate configuration of the porous silicon film and its dust for each drug. The drug conjugated LPSiNPs can be monitored once delivered to a subject. Light intensity from the LPSiNPs can be measured using a low power spectrophotometer. Using such methods the half-life, delivery and collection of drugs and/or LPSiNPs can be monitored.

The luminescent spectrum used in particle identification can readily be measured with inexpensive and portable instrumentation such as a CCD spectrometer or a diode laser interferometer. Removal of a drug from the LPSiNPs can result in a change in the luminescence of the LPSiNPs as a wavelength shift in the spectrum. Such techniques can be used to enable noninvasive sensing through opaque tissue.

The LPSiNS of the disclosure can be formulated for in vitro and in vivo administration using techniques known in the art.

The LPSINS materials of the disclosure can be formulated in pharmaceutically acceptable carrier. Pharmaceutically acceptable carriers useful for administration to a cell, tissue or subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The following examples are intended to illustrate but not limit the disclosure. While they are typical of those that might be used, other procedures known to those skilled in the art may alternatively be used.

### EXAMPLES

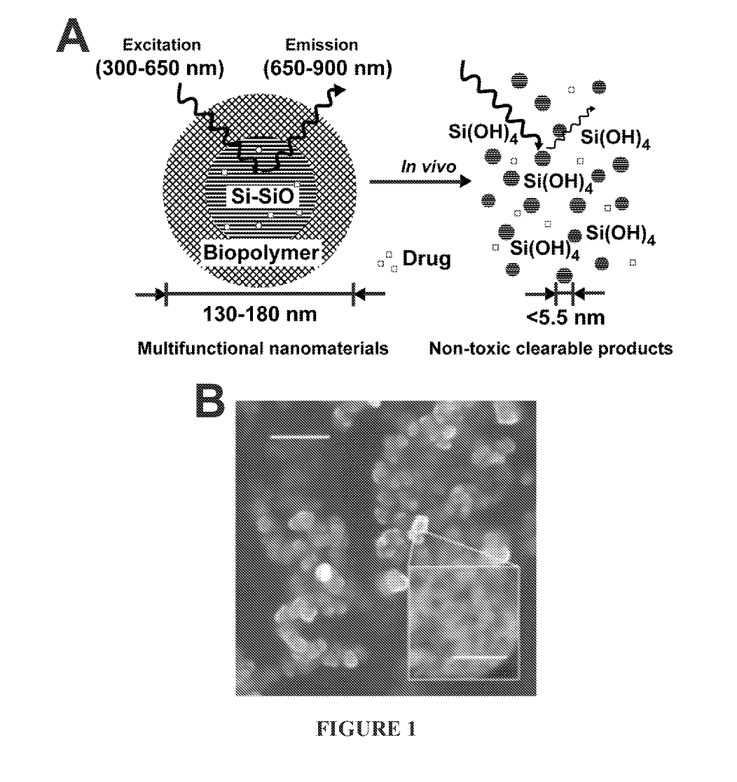

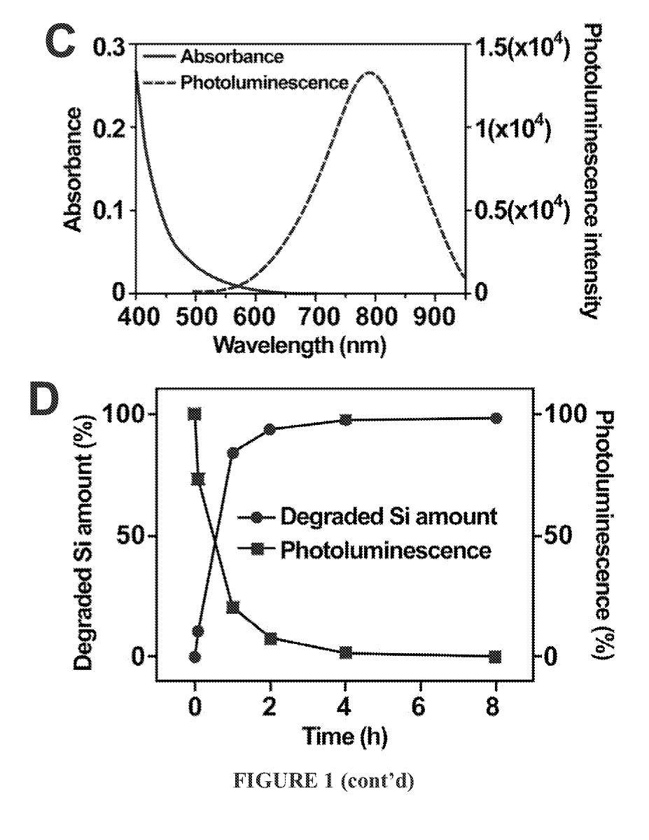

The disclosure demonstrates that porous silicon nanostructures with intrinsic NIR luminescence can be used for in vivo monitoring, they can be loaded with therapeutics, and they can be engineered to resorb in vivo into benign components that clear renally within specific timescales (FIG. 1a).

Luminescent porous Si nanoparticles (LPSiNP) were prepared by electrochemical etching of single-crystal silicon wafers in ethanolic HF solution, lift-off of the porous silicon film, ultrasonication, filtration of the formed particles through a 0.22 μm filtration membrane and finally activation of luminescence in an aqueous solution (FIG. 5). During the activation step, silicon oxide grows on the hydrogen-terminated porous silicon surface, generating significant luminescence attributed to quantum confinement effects and to defects localized at the Si/SiO₂ interface (FIGS. 6 and 7). The preparation conditions were optimized to provide pore volume and surface area suitable for loading of therapeutics and long in vivo circulation times while maintaining an acceptable degradation rate (FIGS. 8 and 9). As a result, the medium-sized (126 nm) particles prepared by electrochemical etching at 200 mA/cm² for 150 s were chosen for the study presented here. The LPSiNP appear spherical and fairly uniform in the scanning electron microscope (SEM), with a well-defined micro- and meso-porous nanostructure (FIG. 1b). The pore diameters are on the order of 5-10 nm (FIG. 8). The mean hydrodynamic size measured by dynamic light scattering (DLS) is ˜126 nm, consistent with the SEM measurements.

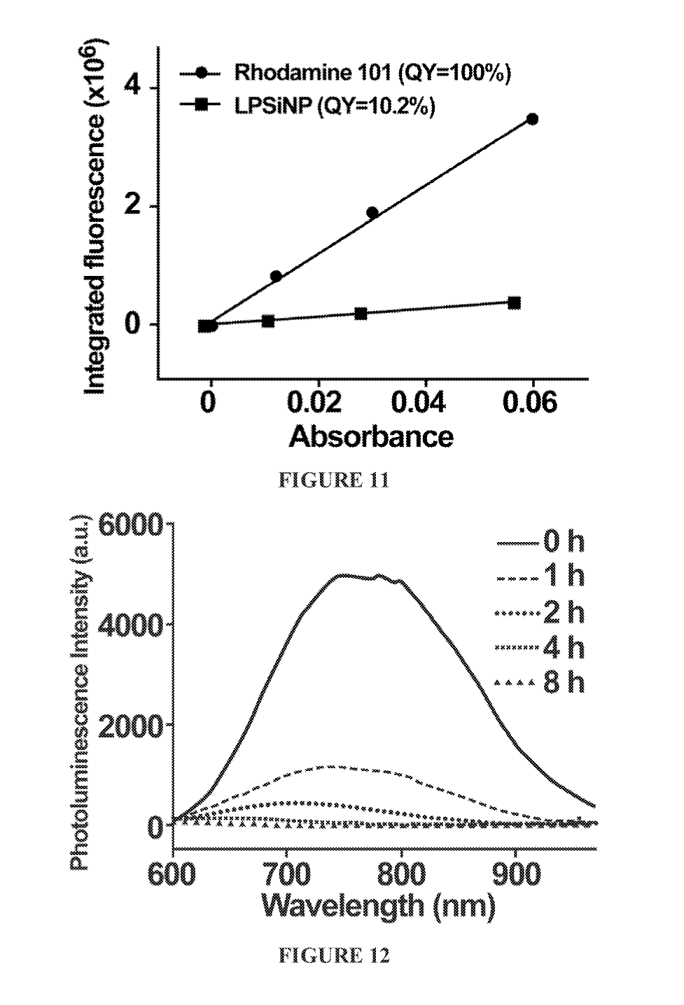

The intrinsic photoluminscence of LPSiNP under UV excitation appears at wavelengths between 650 and 900 nm (FIG. 1c), suitable for in vivo imaging due to low tissue adsorption in this spectral range. The materials display greater photostability relative to fluorescein or the well-known NIR cyanine fluorophores, Cy5.5 and Cy7 (FIG. 10). The quantum yield of LPSiNP in ethanol was determined to be ˜10.2% (relative to Rhodamine 101 standard) (FIG. 11), which is in accord with previously reported values for other water-soluble luminescent silicon/silica nanoparticles. When placed in biological solution (phosphate buffered saline, PBS, pH 7.4, 37° C.) at a mass concentration less than the solubility of silicic acid (0.1˜0.2 mg/mL SiO₂), LPSiNP lose their luminescence in a short time and dissolve (FIG. 1d). A blue-shift of the luminescence spectrum during degradation is indicative of a shrinking in size of the semiconductor fluorophore (FIG. 12). No detectable (by DLS) LPSiNP remain after 8 h of incubation. However, degradation is slowed by addition of a molecular or polymeric surface coating (see below).

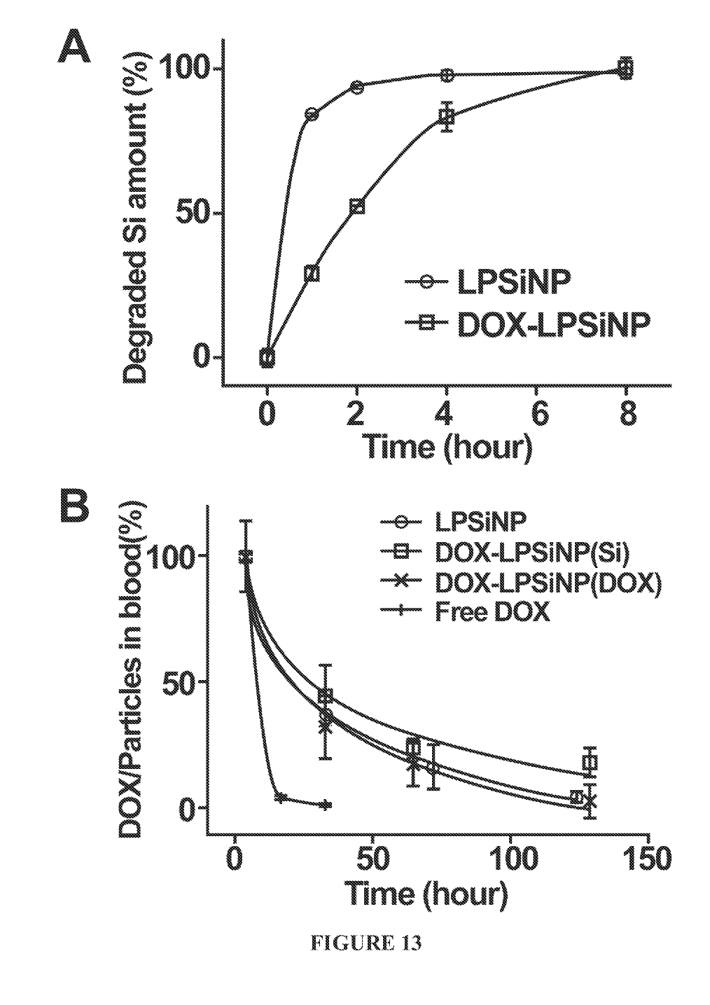

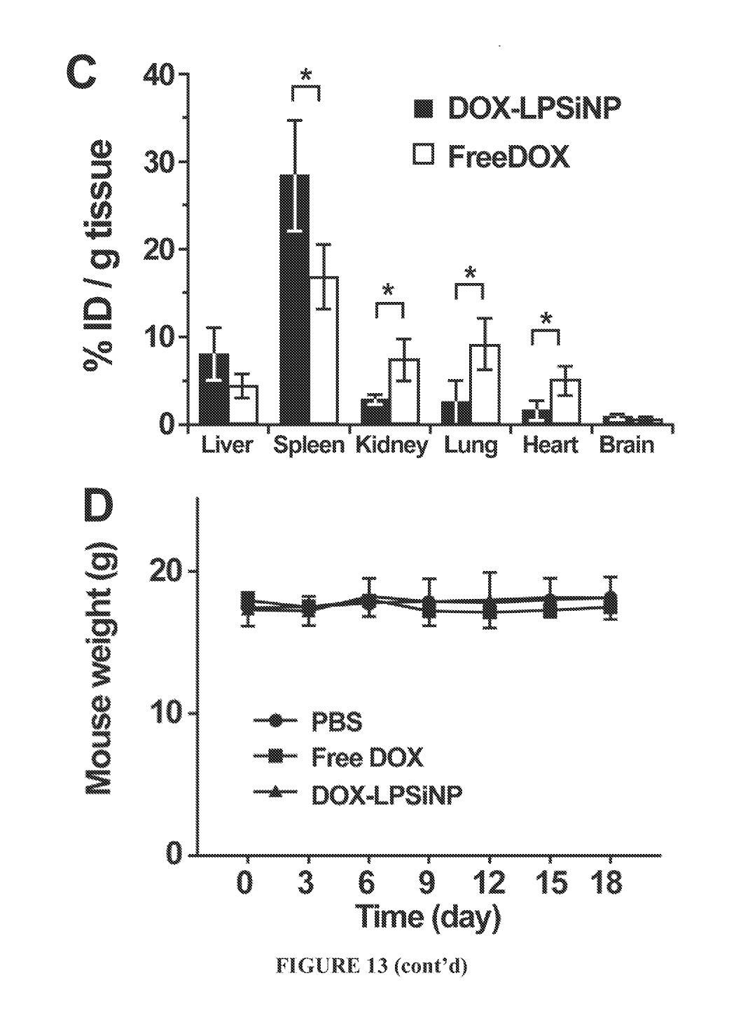

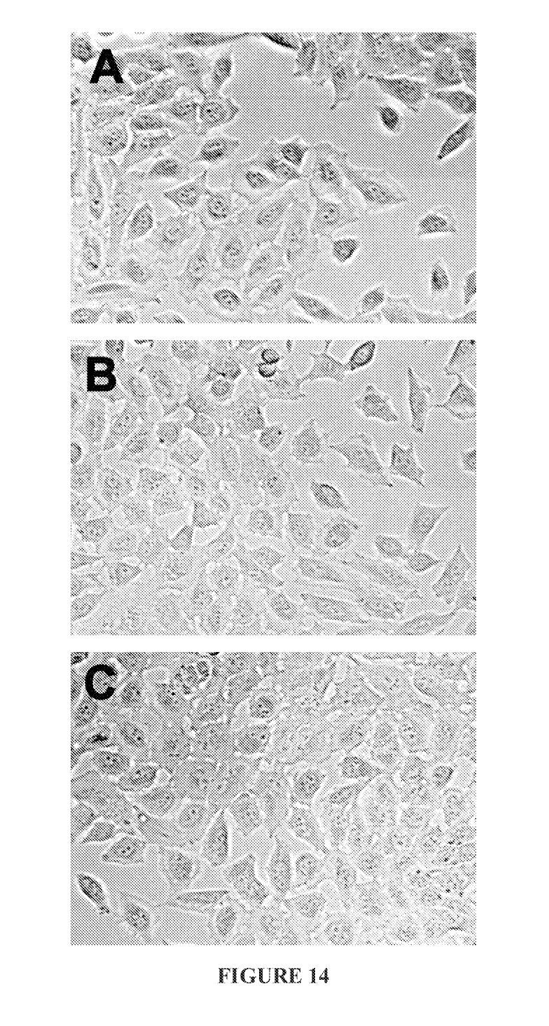

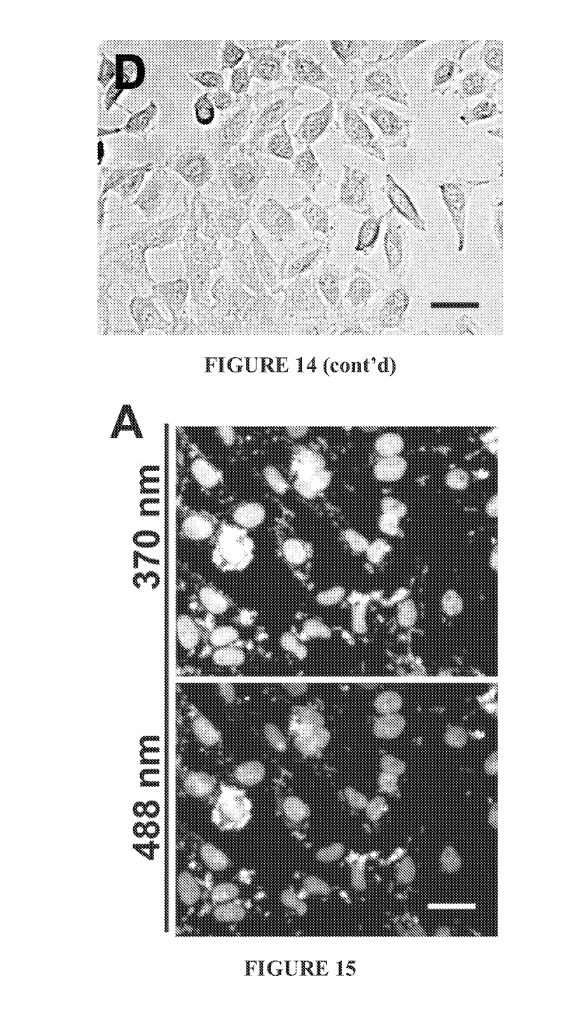

The anti-cancer drug doxorubicin (DOX) was incorporated into the LPSiNP (DOX-LPSiNP, ˜43.8 ug DOX per 1 mg LPSiNP) to test their potential for therapeutic applications (FIG. 13). The positively charged DOX molecules are bound to the negatively charged porous SiO₂ surface by electrostatic forces. Loading of DOX increases the zeta potential of the nanoparticles from −52 mV to −39 mV. A relatively slow release of the drug is observed at physiological pH and temperature, reaching significant levels within 8 h (FIG.. 1e). The appearance of free silicic acid in solution as a function of time, indicative of degradation of the LPSiNP, correlates with the DOX release profile. The rate of degradation of DOX-LPSiNP is somewhat slower than bare LPSiNP (FIG. 13). The presence of DOX molecules inhibits the nanoparticle dissolution process by slowing the rate of SiO₂ hydrolysis at the LPSiNP surface.

DOX-LPSiNP exhibit similar or slightly greater cytotoxicity relative to free DOX, while bare LPSiNP show no significant cytotoxicity (FIG. 1f). It is possible that silicic acid released by the LPSiNP increases the cytotoxicity of DOX by decreasing local extracellular or intracellular pH. In a preliminary in vivo study (FIG. 13), DOX-LPSiNP displayed similar circulation times to bare LPSiNP, suggesting that the attached DOX molecules have no significant effect on LPSiNP circulation, in contrast to the rapid clearance observed with nanoparticles that are coated with positively charged polymer/peptide. Importantly, DOX-LPSiNP retained DOX molecules during circulation and delivered them to organs related to nanoparticle clearance such as the liver and the spleen. Previous work has shown that sequestration of DOX (in that case, in liposomes) reduces cardiotoxicity by reducing the systemic concentration of free DOX.

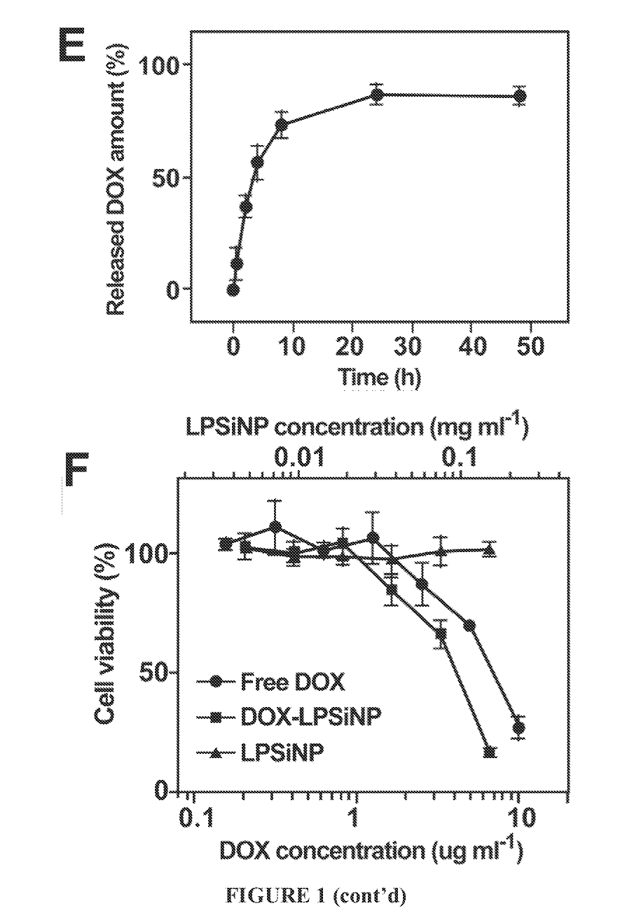

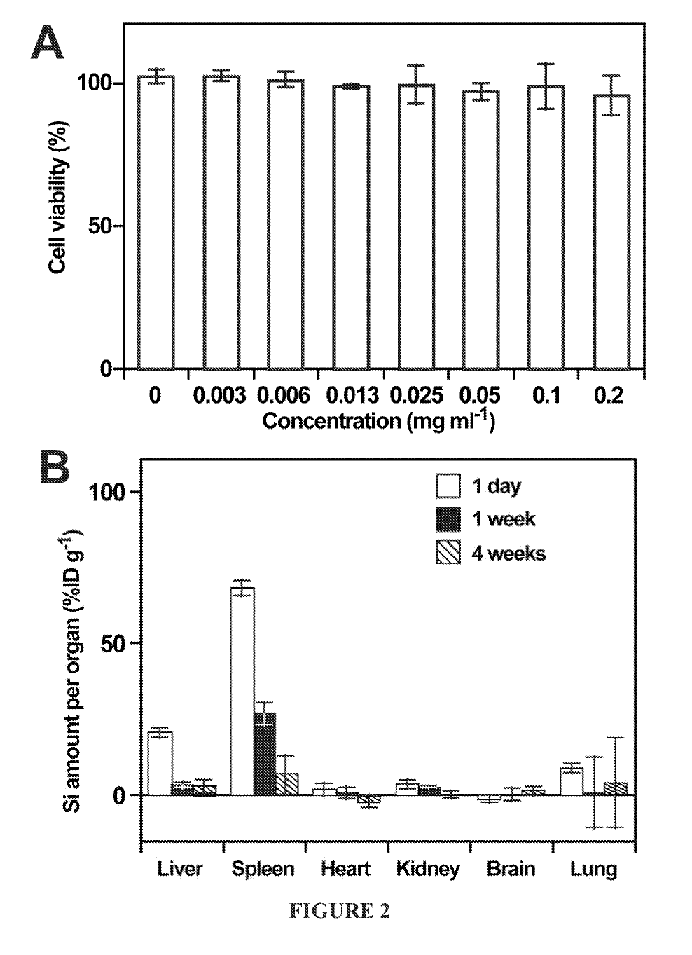

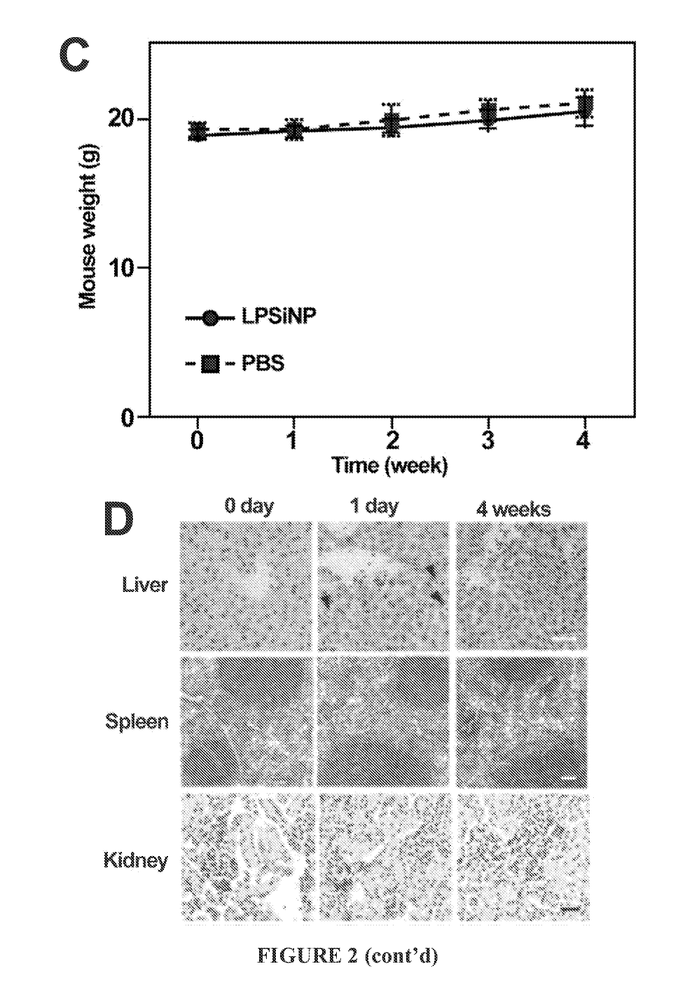

Next, biodegradability and biocompatibility of LPSiNP were tested in vitro and in vivo. The LPSiNP formulation is relatively non-toxic to HeLa cells in vitro within the tested concentration range (FIG. 2a and FIG. 8, 9, 14). For in vivo studies, LPSiNP (20 mg/kg) were injected intravenously into mice. As with many other nanomaterials, the injected LPSiNP accumulate mainly in the MPS-related organs such as the liver and the spleen (FIG. 2b). However, the LPSiNP accumulated in the organs are noticeably cleared from the body within a period of 1 week and completely cleared in 4 weeks. The mechanism of clearance is attributed to degradation into soluble silicic acid followed by excretion. This result contrasts with the slow clearance generally observed for other types of inorganic nanoparticles with diameters>5.5 nm. Over a period of 4 weeks, the body weight of the mice injected with LPSiNP increased slightly in a pattern similar to the control mice (FIG. 2c), indicating that the mice continue to mature without any significant toxic effects.

Since the degradation of highly localized LPSiNP may induce subsequent damage in the organs related to nanoparticle clearance, in vivo toxicity of LPSiNP was further examined in kidney, liver, and spleen tissues of mice 1 day and 4 weeks after LPSiNP injection. Histopathologically, no significant toxicity was observed in these tissues relative to the controls (FIG. 2d). Hepatocytes in the liver samples appeared unremarkable, and there were no inflammatory infiltrates. However, the sinusoids in between the rows of hepatocytes contained Kupffer cells (macrophages) that appeared swollen 1 day after injection. The cells returned to the normal morphology 4 weeks after injection, implying that LPSiNP were taken up, degraded (presumably by lysosomes), and the soluble products were subsequently released from the cells. Spleen samples showed no significant change in morphology of the lymphoid follicles or in the size of the red pulp after LPSiNP injection. Kidney samples also showed no remarkable change in the morphology. Although the in vivo toxicity results shown here are preliminary, the LPSiNP show promise as non-toxic biodegradable inorganic nanomaterials.

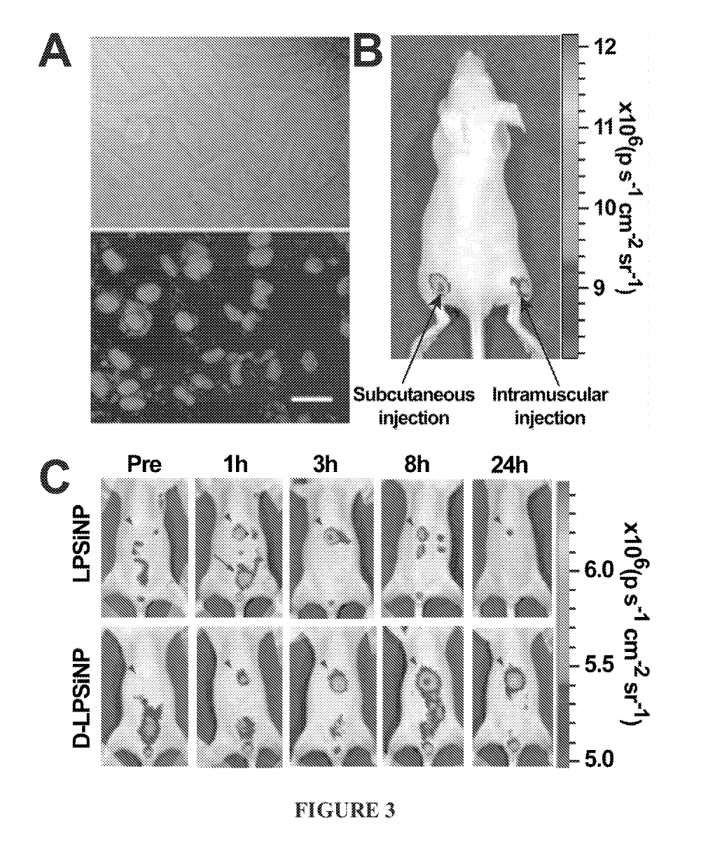

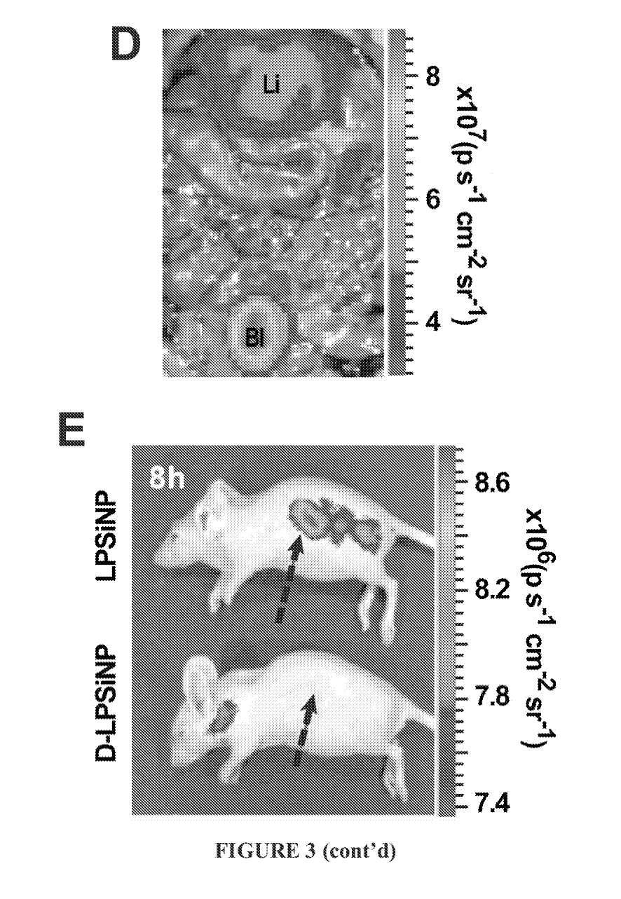

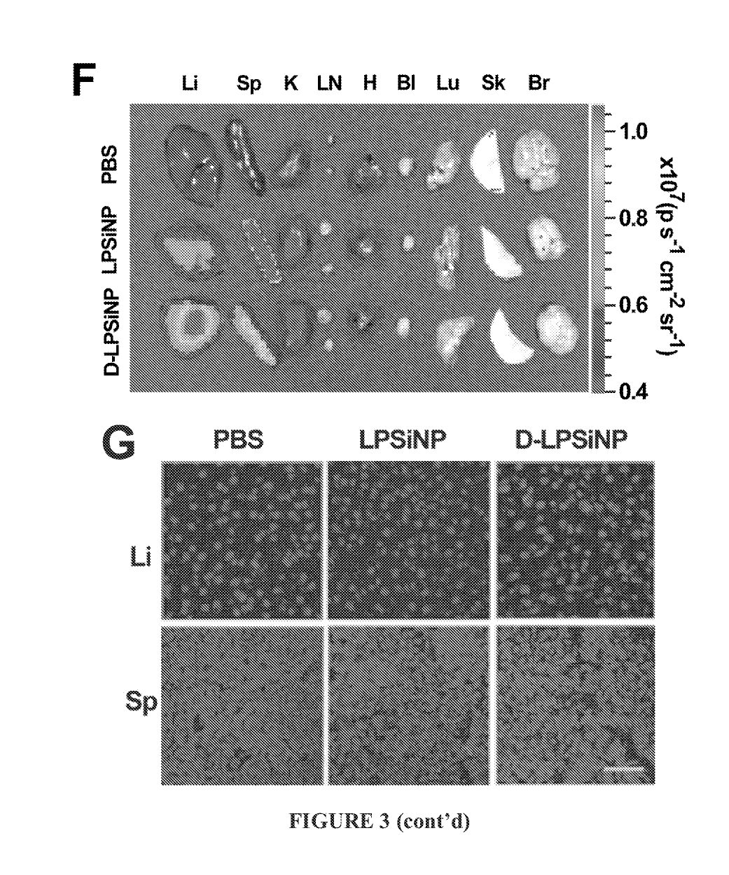

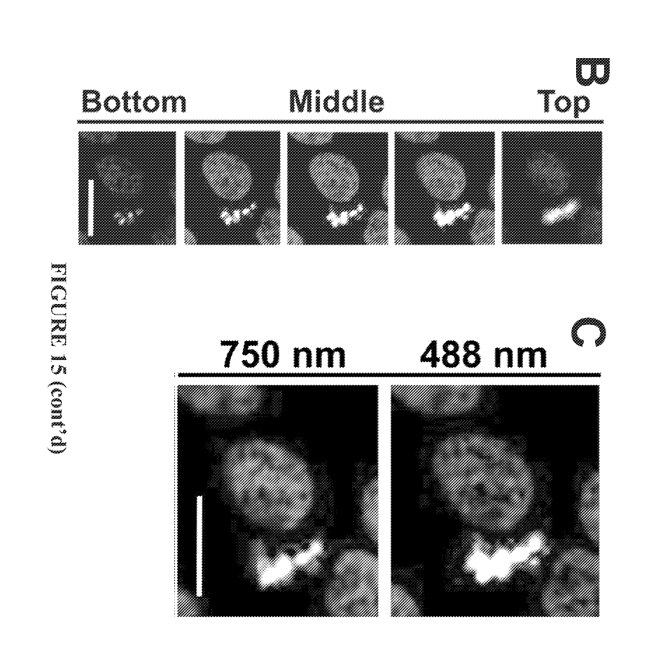

The possibility of imaging cells in vitro and organs in vivo using the intrinsic photoluminescent properties of LPSiNP were examined. Significant luminescence of LPSiNP was observed in HeLa cells using excitation wavelengths of 370 nm, 488 nm, and 750 nm (two-photon excitation) 2 hr after incubation, attributed to non-specific cellular uptake of the silica-based nanomaterials (FIG. 3a and FIG. 15). To examine their potential for in vivo imaging, subcutaneous and intramuscular injections of LPSiNP dispersions (20 μL aliquots, 0.1 mg/mL) into the left and right flank of a nude mouse, respectively, were administered. The mouse was imaged in a fluorescence mode (GFP excitation filter, 445-490 nm and ICG emission filter, 810-875 nm). The signals from both injections were clearly observed without any skin autofluorescence although the near-skin fluorescence intensity is larger than the signal emanating from deeper tissue (FIG. 3b). The fluorescence spectrum of LPSiNP allows imaging in the NIR-emission range, a convenient window for in vivo imaging due to the low levels of NIR autofluorescence of mouse skin excited with visible light.

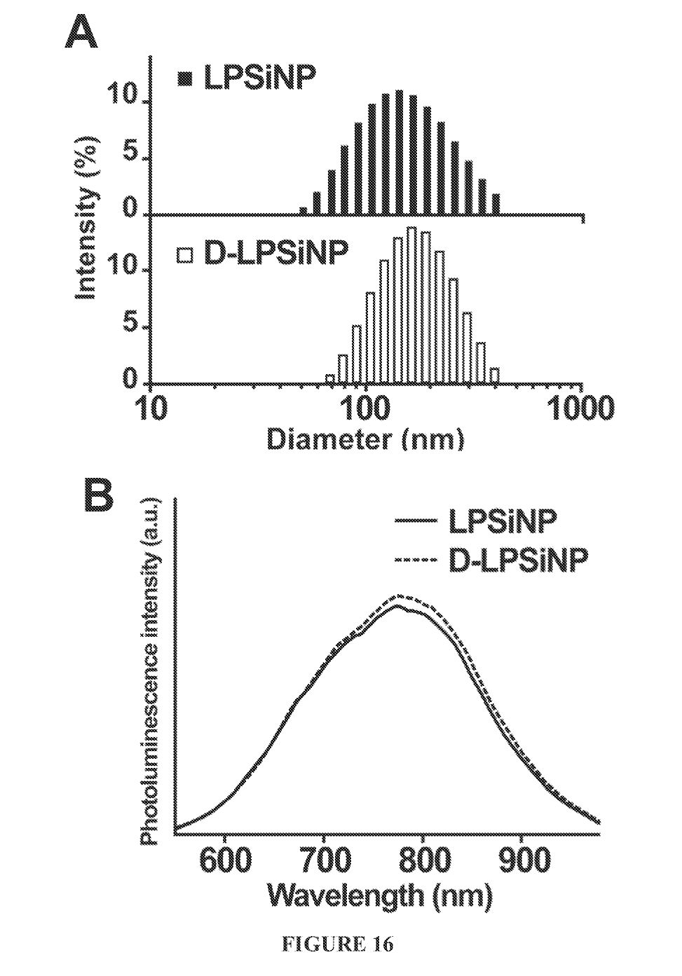

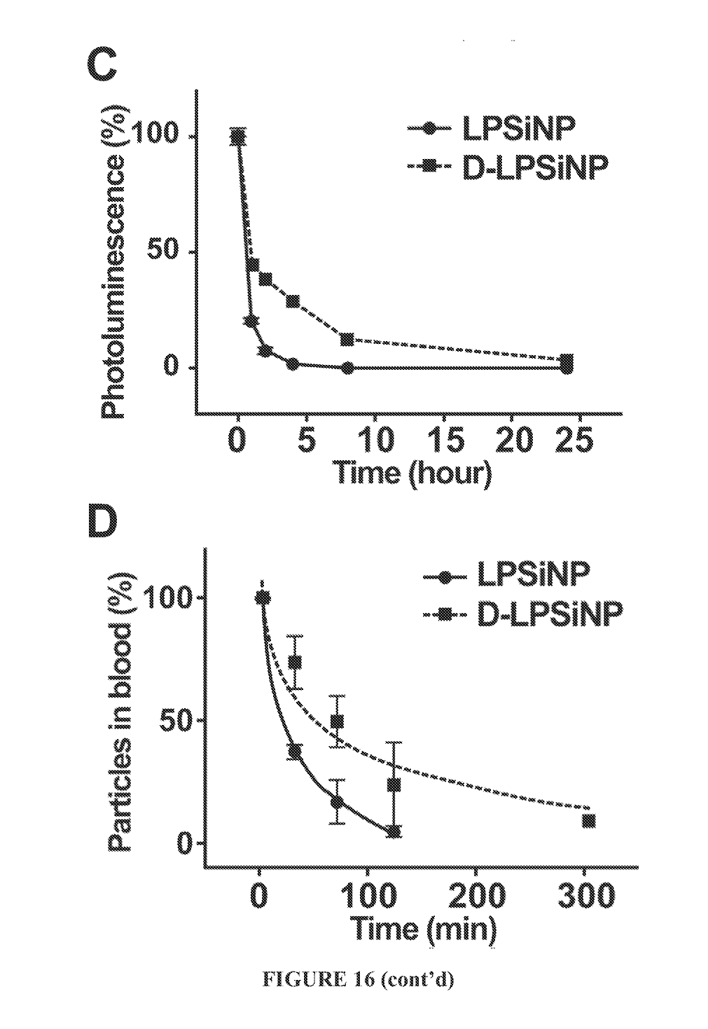

Whole-body fluorescence imaging of nude mice using LPSiNP administered by intravenous injection was performed. To prevent rapid degradation after injection and to increase their blood half-life, LPSiNP were coated with the biopolymer dextran by physisorption (D-LPSiNP, FIG. 16). The coating process increased the size and zeta potential of the nanoparticles (from 125 nm to 151 nm and from −52 mV to −43.5 mV, respectively). Bare LPSiNP or D-LPSiNP were injected and imaged at different time points (FIGS. 3c, 3d, and 3e). A significant fraction of the bare LPSiNP were immediately removed by renal clearance, presumably due to their degradation into smaller (<5.5 nm) nanoparticles. The remaining nanoparticles were observed to accumulate in the liver and the spleen, consistent with the histology data discussed above.

The D-LPSiNP exhibit a somewhat different pattern in their uptake by the MPS-related organs. These nanoparticles accumulate and degrade in the liver slowly relative to bare LPSiNP, which is consistent with the in vitro degradation and in vivo blood half-life data (FIG. 16). Biodistribution and histological studies of the organs harvested from the same mice 24 h after injection are consistent with the whole-body fluorescence imaging data (liver<spleen for LPSiNP and liver>spleen for D-LPSiNP) (FIGS. 3f and 3g). These results indicate that the intrinsic luminescent properties of LPSiNP allow the non-invasive monitoring of their biodistribution and degradation in a live animal as well as the microscopic observation of their localization in the organs.

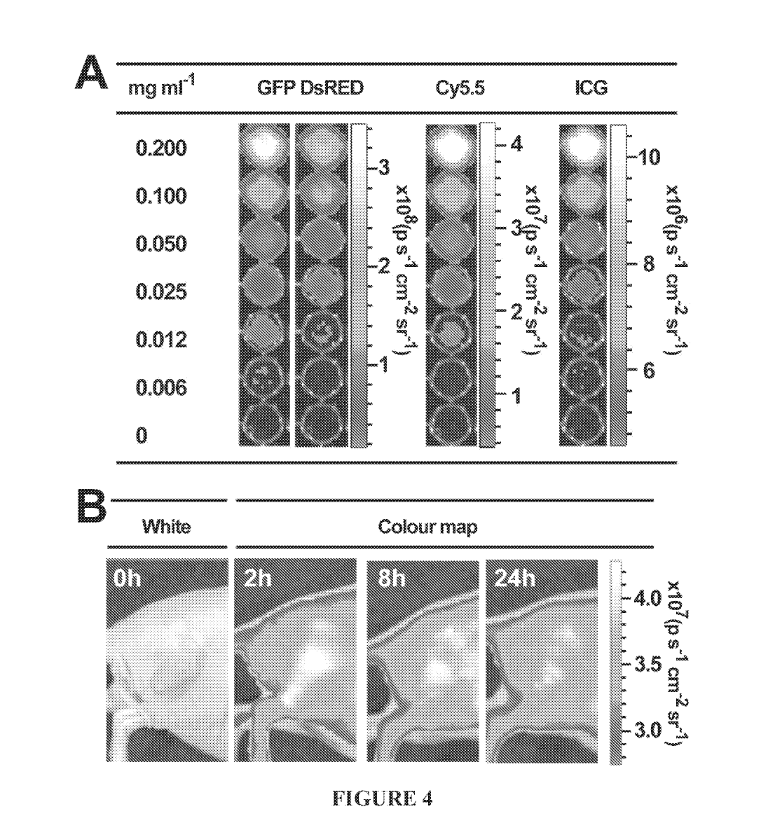

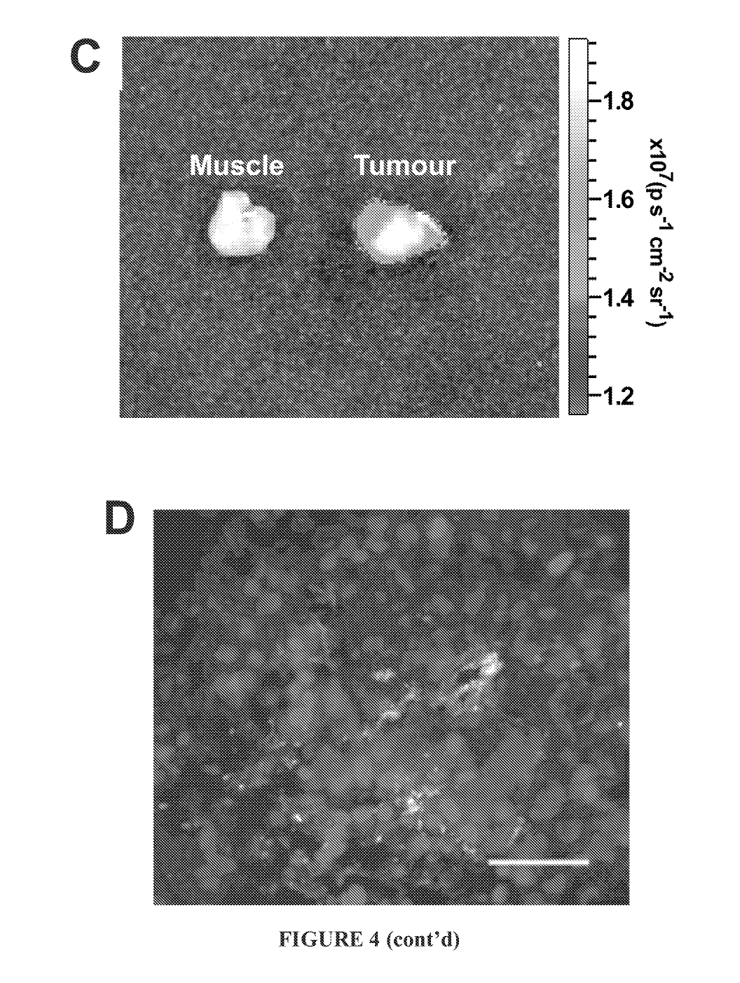

The potential of LPSiNP to image tumors in vivo was evaluated. In order to detect and image deep-tissue diseases such as tumors by fluorescence, the excitation wavelength for the nanoparticle should be in the NIR range in order to maximize tissue penetration and minimize optical absorption by physiologically abundant species such as hemoglobin. LPSiNP emit in the NIR (810-875 nm) and they can be excited with red or NIR radiation (about 300-665 nm by single photon excitation or about 700-900 nm by two photon excitation) (FIG. 4a) or by two-photon NIR excitation (FIG. 15). Similar to some of the NIR-emitting semiconductor QD, the quantum efficiency of LPSiNP decreases with longer excitation wavelengths. However, the quantum yield is sufficient to allow their observation in internal organs using a conventional fluorescence imaging system.

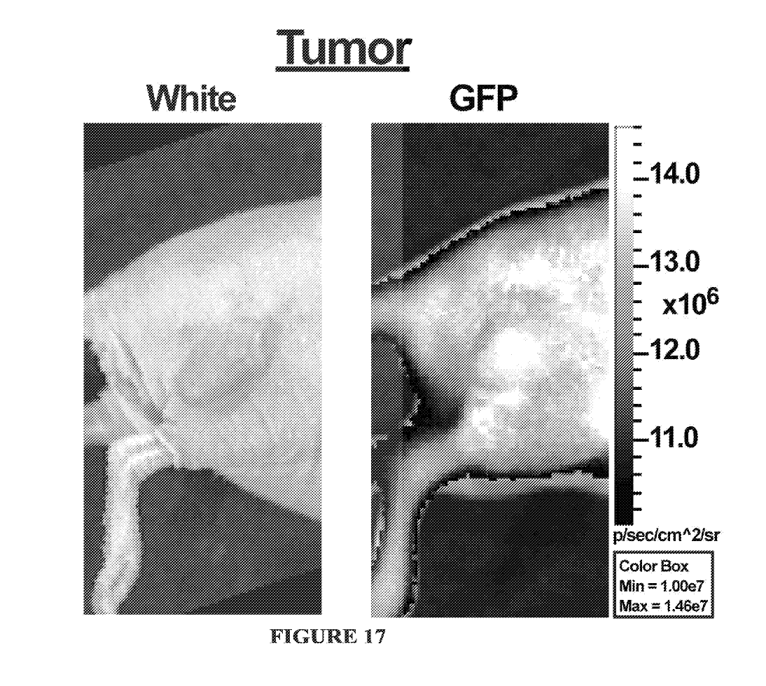

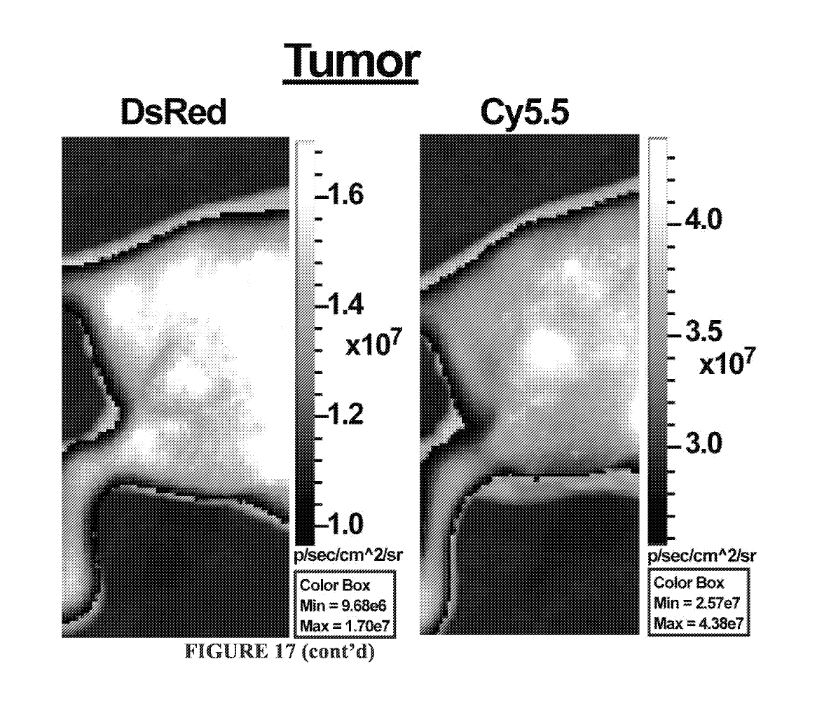

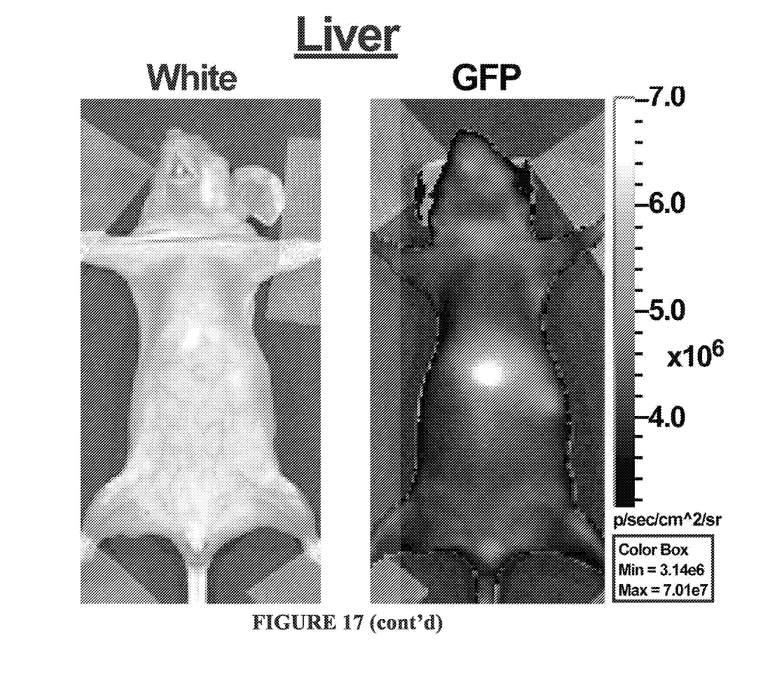

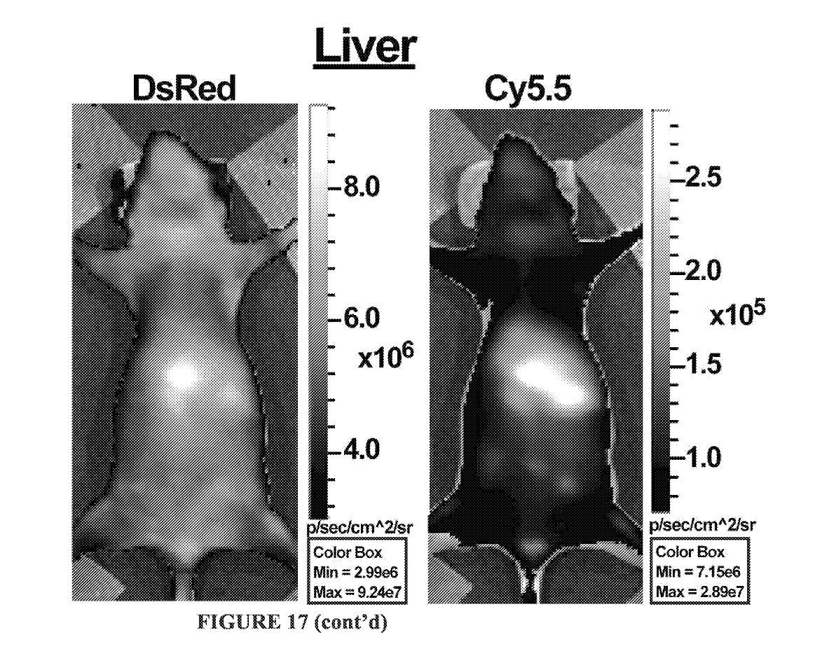

Injection of the D-LPSiNP formulation (20 mg/kg) into a nude mouse bearing an MDA-MB-435 tumor results in passive accumulation of the nanomaterial in the tumor, as revealed in the NIR fluorescence image (FIG. 4b). Imaging with shorter excitation wavelengths (blue or green filter sets) results in poor differentiation of the target organ relative to the surrounding skin area (FIG. 17). The ex vivo fluorescence images and histology confirm the presence of D-LPSiNP in the tumor (FIGS. 4c and 4d).

Thus, the disclosure demonstrate the imaging of a tumor and other organs using biodegradable silicon nanoparticles in live animals, and it is important because of the biodegradability and low in vivo toxicity observed. The LPSiNP injected intravenously are observed to accumulate mainly in MPS-related organs and are degraded in vivo into apparently non-toxic products within a few days and removed from the body through renal clearance. These larger (100 nm-scale) silicon-based biodegradable nanoparticles overcome many of the disadvantages of smaller (<5.5 nm) nanocrystals such as fast clearance from circulation, low capacity for drug loading, and toxicity of the residual particles that do not escape MPS uptake.

### METHODS

Preparation of luminescent porous silicon nanoparticles (LPSiNP). Porous silicon samples were prepared by electrochemical etch of a p-type silicon wafer by application of a constant current density of 200 mA/cm² for 150 s in an aqueous HF/ethanol electrolyte. A freestanding film of the porous silicon nanostructure was then removed from the crystalline silicon substrate by application of a current pulse of 4 mA/cm² for 250 s in an aqueous HF/ethanol electrolyte. The freestanding hydrogen-terminated porous silicon film was fractured by sonication overnight, and then filtered through a 0.22 μm filtration membrane (Millipore). The nanoparticles were further incubated in deionized (DI) water for ˜2 weeks to activate their luminescence in the near-infrared range. For dextran-coated LPSiNP (D-LPSiNP), dextran (MW˜20,000, Sigma) was physically absorbed on LPSiNP.

Porous silicon samples were prepared by electrochemical etch of a single-crystal, (100)-oriented p-type silicon wafer (0.8-1.2 mΩ cm, Siltronix) by application of a constant current density of 200 mA/cm² for 150 s in a 3:1 (v/v) electrolyte of 48% aqueous HF/ethanol. A freestanding film of the porous silicon nanostructure was then removed from the crystalline silicon substrate by application of a current pulse of 4 mA/cm² for 250 s in a solution of 3.3% (by volume) 48% aqueous HF in ethanol. The freestanding hydrogen-terminated porous silicon film was placed in deionized (DI) water and fractured into multi-sized particles by sonication overnight. The particles were then filtered through a 0.22 μm filtration membrane (Millipore). The nanoparticles were further incubated in DI water for ˜2 weeks to activate their luminescence in the near-infrared range. Finally, in order to remove dissolved silicic acids during the activation and obtain porous silicon nanoparticles in a size range of 20-200 nm, the activated nanoparticles were spun down in DI water at 14,000 rpm for 30 min, the supernatant containing silicic acids and smaller nanoparticles (<20 nm) was removed.

For LPSiNP with different porous nanostructures, porous Si samples were prepared by electrochemical etch of a single-crystal, (100)-oriented p-type silicon wafer (0.8-1.2 mΩ cm, Siltronix) by application of a constant current density of 50 mA/cm² for 300 s, 200 mA/cm² for 150 s or 400 mA/cm² for 150 s in a 3:1 (v/v) electrolyte of 48% aqueous HF/ethanol. A freestanding film of the porous silicon nanostructure was then removed from the crystalline silicon substrate by application of a current pulse of 4 mA/cm² for 250 s in a solution of 3.3% (by volume) 48% aqueous HF in ethanol. The freestanding hydrogen-terminated porous silicon film was placed in deionized (DI) water and fractured into multi-sized particles by sonication overnight. The particles were then filtered through a 0.22 μm filtration membrane (Millipore). The nanoparticles were further incubated in DI water for ˜2 weeks to activate their luminescence in the near-infrared range. Finally, in order to remove dissolved silicic acid and obtain porous silicon nanoparticles in a size range of 20-200 nm, the activated nanoparticles in DI water were spun down at 14,000 rpm for 30 min, the supernatant containing silicic acid and non-porous smaller nanoparticles (<20 nm) was removed.

For LPSiNP with different sizes, the particles were then filtered through a 0.45 μm filtration membrane (Millipore) after overnight sonication process. The nanoparticles were further incubated in DI water for ˜2 weeks to activate their luminescence in the near-infrared range. First, LPSiNP with larger sizes (hydrodynamic size=˜270.3 nm) were obtained by centrifugation at low speed (at 6,000 rpm for 10 min) and removal of the supernatant. The nanoparticles were then filtered through a 0.22 μm filtration membrane. Secondly, LPSiNP with medium size (hydrodynamic size=˜125.7 nm, which are the LPSiNP used mainly in this study) were obtained by centrifugation at high speed (at 14,000 rpm for 30 min) and removal of the supernatant. Lastly, LPSiNP with smaller size (hydrodynamic size=˜14.5 nm) were obtained from the supernatant.

To prepare D-LPSiNP, a dextran coating was applied. A 1 mL aliquot of an aqueous dispersion of 0.5 mg of LPSiNP was mixed with a 1 mL aliquot of water containing 100 mg of dextran (MW˜20,000, Sigma). The mixture was stirred overnight, rinsed three times using a centrifugal filter (100,000 Da molecular weight cut-off, Millipore, inc.), the particles were resuspended in water and then filtered through a 0.22 μm filtration membrane.

Nanoparticle characterization. Scanning electron micrographs (SEM) were obtained with a Hitachi S-4800 field-emission instrument. Dynamic light scattering (Zetasizer Nano ZS90, Malvern Instruments) was used to determine hydrodynamic size and zeta potential of (D-)LPSiNP. The photoluminescence (PL, λₑₓ=370 nm and 460 nm longpass emission filter) and absorbance spectra of (D-)LPSiNP were obtained using a Princeton Instruments/Acton spectrometer fitted with a liquid nitrogen-cooled silicon charge-coupled device detector, and a Hewlett-Packard 8452A UV-vis diode array spectrophotometer, respectively. Fluorescence images of D-LPSiNP subjected to different excitation wavelength bands were obtained using an IVIS 200 imaging system (Xenogen).

Scanning electron micrographs (SEM) were obtained with a Hitachi S-4800 field-emission instrument. A 20 uL drop of ethanol containing LPSiNP was directly placed onto a polished silicon wafer and the solvent allowed to dry in air. Dynamic light scattering (Zetasizer Nano ZS90, Malvern Instruments) was used to determine hydrodynamic size and zeta potential of LPSiNP or D-LPSINP (in DI water). To analyze porous nanostructure (pore surface area, pore size, and pore volume) of LPSiNP, N₂ adsorption isotherms (interpreted with the BJH and BET models) were measured on a Micromeritics Accelerated Surface Area and Prosimetry analyzer (ASAP 2020).

The photoluminescence (PL, λₑₓ=370 nm and 460 nm longpass emission filter) and absorbance spectra of LPSiNP or D-LPSiNP in DI water were obtained using a Princeton Instruments/Acton spectrometer fitted with a liquid nitrogen-cooled silicon charge-coupled device detector, and a Hewlett-Packard 8452A UV-vis diode array spectrophotometer, respectively. Fluorescence images of D-LPSiNP in DI water subjected to different excitation wavelength bands were obtained using an IVIS 200 imaging system (Xenogen). (GFP: 445-490 nm and 1 s exposure time, DsRed: 500-550 nm, 2 s exposure time, Cy5.5: 615-665 nm, 8 s exposure time, and ICG: 710-760 nm, 20 s exposure time). The emission filter used was ICG (810-875 nm).

The photostability (photobleaching) of LPSiNP was evaluated relative to organic dyes commonly used in biological imaging (fluorescein, Cy5.5 and Cy7). The LPSiNP and dyes (dispersed or dissolved in aqueous solution) were illuminated with a 100 W mercury lamp, and fluorescence intensities were monitored using a fluorescence microscope (Nikon Eclipse LV150) equipped with a thermoelectrically cooled CCD camera (Photometrics CoolSNAP HQ²). Excitation (355±25 nm for LPSiNP, 480±20 nm for Fluorescein, 650±22 nm for Cy5.5, and 710±35 nm for Cy7) and emission (435 nm long pass for LPSiNP, 535±25 nm for Fluorescein, 710±25 nm for Cy5.5, and 800±35 nm for Cy7) were used for these experiments. The fluorescence intensities were monitored at 0.5 or 1 min intervals. The quantum yield (QY) of LPSiNP in ethanol was measured using the comparative method, using Rhodamine 101 (QY=100%, Sigma) in ethanol as the standard. The Fourier-transform infrared (FTIR) spectra of as-etched porous silicon films and LPSiNP were obtained in the absorption mode using a Thermo Scientific Nicolet 6700 FTIR spectrometer equipped with a diamond Attenuated Total Reflectance (ATR) accessory.

In vitro degradation. (D-)LPSiNP were incubated at 37° C. in phosphate buffered saline (PBS). An aliquot was removed at different time points and filtered with a centrifugal filter (30,000 Da molecular weight cut-off, Millipore) to remove undissolved LPSiNP. The filtered solution was subjected to analysis by inductively coupled plasma optical emission spectroscopy (ICP-OES, Perkin Elmer Optima 3000DV). The decrease in PL of the above samples over time was also monitored.

A series of samples containing 0.05 mg/mL of LPSiNP or D-LPSiNP in 1 mL of PBS solution were incubated at 37° C. An aliquot of 0.5 mL of solution was removed at different time points and filtered with a centrifugal filter (30,000 Da molecular weight cut-off, Millipore, inc.) to remove undissolved LPSiNP. 0.4 mL of the filtered solution was diluted with 4.6 mL HNO₃ (2% (v/v)) and subjected to analysis by inductively coupled plasma optical emission spectroscopy (ICP-OES, Perkin Elmer Optima 3000DV). The silicon concentration in the original solution was determined by incubating the solution in PBS at 37° C. for 72 h and measuring the silicon concentration without filtration. The decrease in PL of the above samples over time was also monitored.

Drug loading and cytotoxicity. LPSiNP was loaded with doxorubicin (DOX, Sigma) in DI water and then rinsed using a centrifugal filter. Release kinetics of DOX from DOX-loaded LPSiNP (DOX-LPSiNP) in PBS at 37° C. was measured by filtering out DOX-LPSiNP from the solution at each time point using the centrifugal filter and measuring fluorescence of free DOX left in the solution at 590 nm (λₑₓ=480 nm). For drug-mediated cytotoxicity experiments, MDA-MB-435 human carcinoma cells were incubated with LPSiNP, DOX-LPSiNP or free DOX for 48 h. The cytotoxicity of LPSiNP, DOX-LPSiNP or free DOX was evaluated using the MTT assay (Chemicon).

0.5 mg LPSiNP (0.5 mg/mL) was mixed with 0.05 mg doxorubicin (DOX, Sigma) in DI water at room temperature overnight and then rinsed three times using a centrifugal filter (100,000 Da molecular weight cut-off, Millipore, inc.). The amount of DOX incorporated into LPSiNP was determined by incubating DOX-loaded LPSiNP (DOX-LPSiNP) in a 0.3 M HCl 70% ethanol solution overnight and comparing the fluorescence with a standard curve (˜43.8 μg DOX per 1 mg LPSiNP). Release kinetics of DOX from DOX-LPSiNP (0.05 mg/mL) in PBS at 37° C. was measured by filtering out DOX-LPSiNP from the solution at each time point using the centrifugal filter and measuring fluorescence of free DOX left in the solution at 590 nm (λₑₓ=480 nm).

For drug-mediated cytotoxicity experiments, MDA-MB-435 human carcinoma cells were incubated with LPSiNP, DOX-LPSiNP or free DOX (at different DOX/LPSiNP concentrations) for 48 h and rinsed with cell medium three times. The cytotoxicity of LPSiNP, DOX-LPSiNP or free DOX was evaluated using the MTT assay (Chemicon). For nanostructure- or size-related cytotoxicity experiments (without DOX), HeLa cells were incubated with the LPSiNP (at different LPSiNP concentrations) for 48 h and rinsed with cell medium (no phenol red) three times. The cytotoxicity of LPSiNP was evaluated using the Calcein assay [fluorogenic intracellular esterase sensor Calcein acetoxymethylester (Calcein AM), Invitrogen]. Cell viability was expressed as the percentage of viable cells compared with controls (cells treated with PBS). The cytotoxicity of the LPSiNP was also examined by observing morphology of live cells using an inverted optical microscope (Nikon).

In vivo degradation, toxicity, and circulation. All animal work was performed in accordance with the institutional animal protocol guidelines in place at the Burnham Institute for Medical Research, and it was reviewed and approved by the Institute's Animal Research Committee. (D-)LPSiNP were intravenously injected into BALB/c mice (20 mg/kg). For in vivo degradation studies, the mice were sacrificed 1 day, 1 week and 4 weeks after injection, and the brain, heart, kidney, liver, lung, and spleen were collected. The tissues were weighed, digested, and then analyzed for silicon content using ICP-OES. For the in vivo toxicity studies, the mass of each mouse was monitored for 4 weeks after injection and compared with control mice (PBS-injected). The sections of kidney, liver, and spleen tissues harvested from the mice 1 day and 4 weeks after injection were stained with haematoxylin and eosin and then examined by a pathologist.

Free DOX or DOX-LPSiNP (in 200 μL PBS solution) were intravenously injected into BALB/c mice at a dose of 2 mg DOX/kg body mass (45.5 mg/kg for LPSiNP of DOX-LPSiNP). To examine preliminary in vivo toxicity, body mass of the mice was monitored every 3 days over a period of 3 weeks.

To determine blood half-lives, blood (100 μL) was collected from the periorbital plexus at several different times after injection using heparinized capillary tubes (Fisher), and then immediately mixed with 100 μL of 10 mM EDTA (in PBS) to prevent coagulation. For silicon concentration in the blood (DOX-LPSiNP), the blood samples were digested and prepared as mentioned for the organs. The total silicon concentration in the blood was measured using ICP-OES. For DOX concentration in the blood (free DOX and DOX-LPSiNP), the blood samples were spun down briefly to remove red blood cells and 100 μL of the supernatants was mixed with 100 μL of 0.3 M HCl 70% ethanol solution overnight to extract free DOX molecules from the DOX-LPSiNP. The total DOX concentration in the blood was calculated based on the fluorescence intensity of DOX in the samples (λₑₓ=480 nm and λₑₘ=590 nm).

To determine biodistribution of free DOX and DOX-LPSiNP in mouse, the organs (liver, spleen, kidney, lung, heart, and brain) were collected 24 h after injection, weighed, homogenized in 1.5 mL of 0.3 M HCl 70% ethanol solution, and further incubated in the same solution overnight to extract DOX molecules from the organs and DOX-LPSiNP. The homogenized solution was spun down at 10,000 rpm for 10 min and the supernatant was only used to measure the DOX fluorescence. The DOX fluorescence (λₑₓ=480nm and λₑₘ=590 nm) in each tissue was quantified as % injected dose (% ID) per gram of wet tissue.

In vivo fluorescence imaging. LPSiNP were injected subcutaneously and intramuscularly into the left and right flank, respectively, of a nude mouse, and imaged immediately with GFP excitation (445-490 nm) and ICG (810-875 nm) emission filter using the IVIS 200 imaging system. For systemic administration, (D-)LPSiNP were intravenously injected into nude mice (20 mg/kg). The mice were imaged under anesthesia several different times after injection using the IVIS 200 imaging system. The organs (bladder, brain, heart, kidney, lymph nodes, liver, lung, skin, and spleen), harvested 24 h after injection, were also imaged. The excitation filter used was GFP (445-490 nm) and the emission filter used was ICG (810-875 nm). For in vivo fluorescence tumor imaging, a nude mouse bearing an MDA-MB-435 human carcinoma tumor (˜0.5 cm, one side of flank) was used. The tumor area was imaged under anesthesia several different times after intravenous injection of D-LPSiNP (20 mg/kg) using the IVIS 200 imaging system. The tumor and muscle around the tumor harvested 24 h after injection, were also imaged. The excitation filter used was Cy5.5 (615-665 nm) and the emission filter used was ICG (810-875 nm). For fluorescent histological analysis, sections of liver, spleen, and tumor tissues were fixed with 4% paraformaldehyde, stained with DAPI, and then observed with 370 nm excitation and 650 nm long pass emission filter using the fluorescence microscope.

HeLa cells (3000 cells per well) were seeded into 8-well chamber glass slides (Lab-Tek) and incubated overnight. A 50 pg per well quantity of LPSiNP was added and the cells incubated for 2 h at 37° C. in the presence of 10% fetal bovine serum (FBS). The cells were then rinsed three times with cell medium, fixed with 4% paraformaldehyde for 20 min and then observed in the fluorescence microscope (370 nm or 488 nm excitation and 650 nm long pass emission filter) and in the Radiance 2100/AGR-3Q BioRad Multi-photon Laser Point Scanning Confocal Microscope. For confocal fluorescence microscopy, the cells treated with LPSiNP were imaged using 488 nm Ar ion laser excitation and a 650 nm long pass emission filter. For multi-photon fluorescence microscopy, the cells treated with LPSiNP were imaged using 750 nm Mai-Tai laser excitation. The DAPI and LPSiNP signals were separated using 495 dichroic filter and 560 nm long pass filter.

A number of embodiments of the invention have been described. Nevertheless, it will be understood that various modifications may be made without departing from the spirit and scope of the invention. Accordingly, other embodiments are within the scope of the following claims.

1. A biodegradable porous nanostructure comprising silicon material, an emission spectra of about 500 to about 1000 nm and an excitation spectra between about 290-700 nm by single photon excitation or about 600-1200 nm by two photon excitation.

2. The biodegradable porous nanostructure of claim 2, wherein the silicon material comprises a silicon dioxide material.

3. The biodegradable porous nanostructure of claim 1, comprising a particulate size of between about 5 nm and 100 μm.

4. The biodegradable porous nanostructure of claim 1, wherein the biodegradable porous nanostructure is non-toxic.

5. The biodegradable porous nanostructure of claim 1, coated or encapsulated within a polymeric material.

6. The biodegradable porous nanostructure of claim 5, wherein the polymeric material is dextran, polyethylene glycol (PEG), lipids, chitosan, zein, polylactic acid, polyglycolic acid, collagen, fibrin, co-polymers of polylactic acid and polyglycolic acid, and co-polymers of dextran and polylactic acid.

7. The biodegradable porous nanostructure of claim 5, wherein the polymeric material is dextran.

8. The biodegradable porous nanostructure of claim 1, further comprising a therapeutic drug.

9. A method of making a biodegradable porous nanostructure of claim 1, comprising:
electrochemically etching a p-type silicon wafer;
obtaining a free-standing hydrogen-terminated porous silicon film by removing the porous silicon nanostructure from the crystalline silicon substrates;
fracturing the free-standing hydrogen-terminated porous silicon film to obtain a mixture of nanoporous materials of differing sizes;
filtering or size selecting the fractured porous material to obtain a desired size fractionated nanoporous material; and
activating the size fractionated nanoporous material by incubating the material in an aqueous buffer that is oxidizing or neutral to basic to obtain a luminescent porous silicon nanoparticle (LPSiNP).

10. The method of claim 9, wherein the electrochemical etching is by application of a constant current density of about 200 mA/cm² for about 150 s in an aqueous HF/ethanol electrolyte.

11. The method of claim 9, wherein the freestanding film is obtained by application of a current pulse of about 4 mA/cm² for 250 s in an aqueous HF/ethanol electrolyte.

12. The method of claim 9, wherein the freestanding hydrogen-terminated porous silicon film is fractured by sonication.

13. The method of claim 9, wherein the filtering or size selection comprises passing the nanoporous material through a 0.22-0.45 μm filtration membrane, using chromatography or centrifugation.

14. The method of claim 9, wherein the activating comprises incubating the size fractionated nanoporous material in an aqueous borate buffer.

15. The method of claim 9, wherein the activating comprises incubating the size fractionated nanoporous material in deionized water for approximately 2 weeks.

16. The method of claim 9, further comprising physically absorbing dextran to the LPSiNP.

17. The method of claim 9, further comprising loading a therapeutic drug into the pores of the LPSiNP.

18. A pharmaceutical composition comprising a pharmaceutically acceptable carrier and a biodegradable porous nanostructure of claim 1.

19. A composition comprising:
a biodegradable porous nanostructure comprising silicon, a plurality of pores and comprising an emission spectra of about 500 to about 1000 nm and an excitation spectra between about 290-700 nm by single photon excitation or about 600-1200 nm by two photon excitation; and
a drug or biologically active material within the pores.

20. The composition of claim 19, further comprising a polymeric coating the increases the half-life or circulatory time of the biodegradable porous nanostructure in vivo.

21. A method of preparing a biodegradable imaging agent comprising:
electrochemically etching a p-type silicon wafer;
lifting off a porous film from the silicon wafer substrate;
fractionating the porous film to generate nanostructures;
activating the nanostructure in an oxidizing aqueous buffer.

22. The method of claim 21, wherein the aqueous buffer comprises a borate solution.

23. The method of claim 21, wherein the imaging agent comprises an emission spectra of about 500 to about 1000 nm and an excitation spectra between about 290-700 nm by single photon excitation or about 600-1200 nm by two photon excitation.

24. The method of claim 21, further comprising loading a drug or agent into the pores of the nanostructure.

25. The method of claim 21, further comprising adsorbing a biocompatible agent to the nanostructure to increase the half-life or circulatory time in vivo.

26. A nanostructure made by the method of claim 21.

27. A nanostructure made by the method of claim 22.

28. A nanostructure made by the method of claim 23.

29. A method of imaging a tissue, cell, or tumor comprising administering to a tissue, cell, or subject a nanostructure of claim 1, and contacting the tissue, cell or subject with an excitation energy and measuring an emission spectra.
